(19) United States
(12) Patent Application Publication
(10) Pub. No.: US 2024/0228556 A9
(43) Pub. Date: Jul. 11, 2024

(54) NOVEL CANINE ODORANT BINDING PROTEINS

(71) Applicants: SPECIALITES PET FOOD, ELVEN (FR); Institut national de recherche pour l’agriculture, l’alimentation et l’environnement, PARIS (FR); UNIVERSITE DE BOURGOGNE, DIJON (FR); CENTRE NATIONAL DE LA RECHERCHE SCIENTIFIQUE (CNRS), PARIS (FR); INSTITUT NATIONAL SUPERIEUR DES SCIENCES AGRONOMIQUES, DE L'ALIMENTATION ET DE L'ENVIRONNEMENT, DIJON (FR)

(72) Inventors: Margot GLAZ, DIJON (FR); Fabrice NEIERS, LONGEAULT-PLUVAULT (FR); Loïc BRIAND, SAINT APOLLINAIRE (FR); Emilie LECLERC, SENE (FR); Marion GUILLOUX, LA TRINITE SURZUR (FR)

(73) Assignees: SPECIALITES PET FOOD, ELVEN (FR); Institut national de recherche pour l’agriculture, l’alimentation et l’environnement, PARIS (FR); UNIVERSITE DE BOURGOGNE, DIJON (FR); CENTRE NATIONAL DE LA RECHERCHE SCIENTIFIQUE (CNRS), PARIS (FR); INSTITUT NATIONAL SUPERIEUR DES SCIENCES AGRONOMIQUES, DE L'ALIMENTATION ET DE L'ENVIRONNEMENT, DIJON (FR)

(21) Appl. No.: 18/278,295

(22) PCT Filed: Feb. 23, 2022
(86) PCT No.: PCT/EP2022/054538

§ 371 Date: Aug. 22, 2023

(30) Foreign Application Priority Data
Feb. 23, 2021 (EP) 21305218.6

(51) Int. Cl. C07K 14/47; G01N 33/53

(52) U.S. Cl. CPC C07K 14/47; G01N 33/5308

(57) ABSTRACT

The present invention relates to the use of a sequence chosen among Seq ID No 3, Seq ID No 1, Seq ID No 2, Seq ID No 4, Seq ID No 5, Seq ID No 6, Seq ID No 7, Seq ID No 8, and functional variants thereof, as an odorant binding protein (OBP).

The present invention relates to the field of molecular biology.

More precisely, the present invention relates to the identification of novel canine Odorant Binding Proteins (OBPs) capable of binding hydrophobic compounds, notably odorant compounds. These newly identified OBPs are notably useful for detecting odorant compounds that may be used in the industry.

### BACKGROUND OF THE INVENTION

Lipocalins form a family of proteins, notably found in insects and vertebrates, which have a function in the transport of small hydrophobic molecules such as steroids, bile salts, retinoids and lipids. Lipocalins have been associated with many biological processes, such as immune response, pheromone transport, prostaglandin synthesis, retinoid binding and cancer cell interactions.

This family consists of small proteins (160-190 amino acids) containing a hydrophobic pocket and which are generally secreted although in some cases, proteins remain associated with membranes. Structurally, they share a common tertiary architecture made of a 9 stranded anti parallel beta-barrel linked by a turn to a helical domain followed by a strand of the barrel and the C-terminal tail. The ligand binding site (i.e., the binding pocket) is located in the cavity at the middle of the beta-barrel. However, despite this structural homology, sequence identities between vertebrate lipocalins are low (around 20%).

Odorant binding proteins (usually referred to as “OBPs”) belong to the family of lipocalins. They present a molecular mass of 15-22 kDa and are secreted in the olfactory mucus where the olfactory receptor neurons are bathing. OBP have been identified in many vertebrate species. Like other lipocalins, OBPs typically bind hydrophobic compounds, and preferably odorant compounds, and act as transporters, thereby delivering these compounds to olfactory receptors located in the cell membrane of olfactory neurons. Vertebrate OBPs belong to the lipocalin family and present the same fold. Basically, vertebrate OBPs share a low percentage of identity across and within species. This percentage of conserved residues between species has been shown to be as low as 8% (Tegoni, Campanacci et al. 2004).

Largely studied in many vertebrates (Xenopus, cow, pig, rabbit, mouse, rat, pig, elephant, human beings), OBPs have not been characterized in dogs. For the first time, the present inventors have identified and characterized 4 canine OBPs.

Some of the herein described sequences had previously been identified as lipocalins but their very specific function as an OBP had never been demonstrated, or even described, before.

### SUMMARY

In a first aspect, the present invention relates to the use of a sequence chosen among Seq ID No 3, Seq ID No 1, Seq ID No 2, Seq ID No 4, Seq ID No 5, Seq ID No 6, Seq ID No 7, Seq ID No 8, and functional variants thereof, as an OBP.

In a further aspect, the invention relates to a method of identifying ligands, comprising at least the steps of:
(i) exposing at least one candidate compound to an OBP as defined above;
(ii) detecting whether the candidate compound binds to said OBP; and
(iii) if the candidate compound binds to said OBP then the compound is identified as being a ligand to said OBP.

In another aspect, the present invention also relates to a kit comprising, in one or more containers in a single package:
(i) the OBP as defined in the present invention, and
(ii) one or more candidate compounds.

In particular, such a kit can be used for identifying odorant compounds, such as using the method according to the present invention.

### FIGURES

FIG. 1: Identification of CfamOBPs (for “Canis lupus familiaris OBPs”) by proteomic analysis of olfactory mucus taken from 6 different dogs. For each protein, the Uniprot accession number is indicated. The number of identified peptides and their number of spectra from each of the six analysed dogs is indicated.

FIG. 2: Alignment of the four identified CfamOBPs from Canis lupus familiaris. The numbering used is aligned for the four sequences. Secondary structures are indicated for each OBP with dark grey arrow (B-strands) and light grey strands (α-helices) Signal peptide is indicated in bold and C-terminal α helix is shown in light grey. The sequences were aligned using the BioEdit software (available online).

FIG. 3: SDS-PAGE analysis of the four purified recombinant CfamOBPs. The proteins were visualized using Coomassie blue staining. SDS-PAGE analysis indicates a purity rate over 95%. The different lanes correspond to the different purified CfamOBPs. MW stands for molecular mass marker.

FIG. 4: Fluorescence competitive binding assays on CfamOBP1, CfamOBP2, and CfamOBP3. Fluorescence emission spectra are recorded at 20° C. in presence of 2 μM recombinant OBP. Maximum measured fluorescence is at 470, 400 and 420 nm for CfamOBP1, CfamOBP2 and CfamOBP3, respectively. Competitive binding curves of (A) CfamOBP1-1,8-ANS complex with citronellal (B) CfamOBP2-NPN complex with DMO (3,7-dimethyl-1-octanol) and (C) CfamOBP3-NPN complex with IBMP (2-isobutyl-3-methoxypyrazine).

FIG. 5: Fluorescence competitive binding assays on the variants CfamOBP1-L52 (Seq ID No 14) and CfamOBP2-K83 (Seq ID No 15). Fluorescence emission spectra were recorded at 20° C. with 2 μM of OBP. The maximum fluorescence emission is recorded at 470 and 400 nm for CfamOBP1-L52 and CfamOBP2-K83 respectively. Competitive binding assays of 1,8-ANS with citronellal for CfamOBP1-L52 (A) and of NPN with DMO for CfamOBP2-K83 (B).

FIG. 6: Isothermal titration calorimetry data for binding of IBMP to CfamOBP3 and CfamOBP3-102Y (Seq ID No 35). Binding of IBMP on CfamOBP3 (A) and CfamOBP3-102Y (B), upper panel is the raw data of the ITC experiment with the injection of odorant in the cell containing the OBP. Lower panel represent the binding enthalpy for each injection. Stoichiometry (N) and affinity constants (K_{A}) are indicated under the thermograms.

### DESCRIPTION OF SOME EMBODIMENTS OF THE INVENTION

Unless specifically stated otherwise, percentages are expressed herein by weight of a product reference. In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

As used throughout, the singular form of a word includes the plural, and vice versa, unless the context clearly dictates otherwise. Thus, the references “a”, “an”, and “the” are generally inclusive of the plurals of the respective terms. For example, reference to “a method” includes a plurality of such “methods”. Similarly, the words “comprise”, “comprises”, and “comprising” are to be interpreted inclusively. Likewise, the terms “include”, “including” and “or” should all be construed to be inclusive. All these terms however have to be considered as encompassing exclusive embodiments that may also be referred to using words such as “consist of”.

The methods and compositions and other embodiments exemplified here are not limited to the particular methodologies, protocols, and reagents that are described herein because, as the skilled artisan will appreciate, they may vary.

Unless specifically defined, all technical and scientific terms used herein have the same meaning as commonly understood by a skill artisan in chemistry, biochemistry, cellular biology, molecular biology, and agronomy.

The term “about” as used herein when referring to a measurable value such as an amount, a temporal duration, and the like, is meant to encompass variations of ±20%, more preferably ±10%, even more preferably ±5% from the specified value, as such variations are appropriate to reproduce the disclosed methods and products.

By “isolated” it is meant a molecule, including a protein, polypeptide, peptide, nucleic acid molecule, plasmid vector, viral vector or recombinant cell, which is extracted from its natural environment (i.e., separated from at least one other component with which it is naturally associated).

By “appropriate conditions for”, “suitable conditions for” it is referred to conditions of implementation of a method according to the invention which are either described in the specification, in particular which are illustrated in the Examples, or well-known from a person skilled in the art.

By “canine” it is herein referred to an animal that is a member of the family Caninae, which includes wolves, jackals, foxes, coyote, and the domestic dog (hereafter “dog”). For example, a canine may be a domestic dog, a wolf, or an animal that has some genetic contributions from more than one species of the family Caninae. Preferably, the term “canine” refers to a domestic dog, notably those of the subspecies Canis lupus familiaris.

In the following description, embodiments may be taken alone or combined in an appropriate manner by the skilled person.

While studying the olfactory mucus of dogs, the inventors have surprisingly found that some proteins present in that mucus, which had been referenced in database as lipocalins, could be used as OBPs. These proteins have been renamed CfamOBP1, CfamOBP2, CfamOBP3, and

CfamOBP4. In the context of the invention, these proteins are identified and designated by reference to their sequence ID, i.e., Seq ID No 1, Seq ID No 2, Seq ID No3, and Seq ID No 4, respectively.

A—Use of Isolated Proteins and Polypeptides as OBPs

In a first aspect, the invention relates to the use of a sequence chosen among Seq ID No 3, Seq ID No 1, Seq ID No 2, Seq ID No 4, Seq ID No 5, Seq ID No 6, Seq ID No 7, Seq ID No 8, and functional variants thereof, as an OBP. For the sake of conciseness and clarity, the sequences described and used in the present invention are referred to as “OBPs”.

The present inventors have now identified that the herein described proteins and polypeptides could be used as OBPs which are capable of binding hydrophobic compounds and more particularly, odorant compounds including pheromones.

By “protein” it is meant polymers of amino acid residues which comprise at least nine amino acids linked by peptide bonds. The polymer may be linear, branched or cyclic. The polymer may comprise naturally occurring amino acids and/or amino acid analogues and may be interrupted by non-amino acid residues. As a general indication and without being linked to it in this application, if the amino acid polymer contains more than 50 amino acid residues, it is preferably referred to as a “polypeptide” or “protein”, whereas if the polymer consists of 50 amino acids or less, it is preferably referred to as a “peptide”. Hence in the sense of the present application the terms “protein” and “polypeptide” have similar meaning and can be used interchangeably.

By “binding”, “binds”, it is meant that the proteins and polypeptides used as OBPs form a complex with a binding partner, such as hydrophobic compounds, and preferably odorant compounds. Typically, proteins and polypeptides used as OBPs according to the invention bind at least one odorant comprising a compound such as pyrazine, pyrrole, aldehyde, ketone, ester, alcohol, acid, alkane, alkene, benzene, sulfide, phenol, thiazole, sulphur, hydrocarbon, terpene or terpenoid, furan, furanone, lactone, as well as pheromones. Methods for detecting whether two molecules bind are well known in the art and include, for example, equilibrium dialysis, surface plasmon resonance, ligand competitive binding assays, and the like.

By “hydrophobic molecule” or “hydrophobic compound”, it is meant a molecule which tends to be non-polarized and therefore prefer other neutral molecules and non-polarized solvents. Typically, a hydrophobic molecule is repelled by a mass of water, does not dissolve in water and has no polar affinity with it.

An “odorant”, “odorant compound” or “odorant molecule” includes chemical compounds such as pyrazine, pyrrole, aldehyde, ketone, ester, alcohol, acid, alkane, alkene, benzene, sulfide, phenol, thiazole, sulphur, hydrocarbon, terpene or terpenoid, furan, furanone, lactone, as well as pheromones. Typically, an odorant provides a smell, an odor, a flavor or an aroma, and is sufficiently volatile to be transported to the olfactory system, notably transported by an odorant binding protein to olfactory receptors located in the cell membrane of sensory neurons. For instance, DMO (3,7-dimethyl-1-octanol), B-ionone, citronellal and IBMP (2-isobutyl-3-methoxypyrazine) are considered as odorant molecules.

Polypeptides Used as OBPs

The present invention provides the use of a polypeptide as an OBP. The peptide sequences used as OBPs in the context of the invention are the following:

Seq ID No 1: CfamOBP1
MKTLLLTIGFSLIAILQAQDTPALGKDTVAVSGKWYLKAMTADQEVPEK
PDSVTPMILKAQKGGNLEAKITMLTNGQCQNITVVLHKTSEPGKYTAYE
GQRVVFIQPSPVRDHYILYCEGELHGRQIRMAKLLGRDPEQSQEALEDF
REFSRAKGLNQEILELAQSETCSPGGQ
Seq ID No 2: CfamOBP2
MQLLLLTVGLALICGLQAQEGNHEEPQGGLEELSGRWHSVALASNKSDL
IKPWGHFRVFIHSMSAKDGNLHGDILIPQDGQCEKVSLTAFKTATSNKF
DLEYWGHNDLYLAEVDPKSYLILYMINQYNDDTSLVAHLMVRDLSRQQD
FLPAFESVCEDIGLHKDQIVVLSDDDRCQGSRD
Seq ID No 3: CfamOBP3
MKLLLLCLGLILVHAHEEENDVVKGNFDISKISGDWYSILLASDIKEKI
EENGSMRVFVKDIEVLSNSSLIFTMHTKVNGKCTKISLICNKTEKDGEY
DVVHDGYNLFRIIETAYEDYIIFHLNNVNQEQEFQLMELYGRKPDVSPK
VKEKFVRYCQGMEIPKENILDLTQVDRCLQARQSEAAQVSSAE
Seq ID No 4: CfamOBP4
MRCVLLGQVLVLLWLSGAWAEVLVQPDFDAKKFSGLWYVVSMVSDCKVF
LGKKDHLLMSSRTIRAMPGGNLSVHMEFPRADGCHQLDAEYLRVGSEGH
FRVPALGYLDVRVADTDYDTFAVLYIYKELEGALSTMVQLYSRTQEASP
QATKAFQDFYPTVGLPNDMMVMLPKSDVCSSAGKEAS

In the above identified protein sequences, bold characters represent signal peptides; underlined characters represent consensus sequences and italics characters represent alpha helix.

In an embodiment, the invention relates to the use of a sequence chosen among Seq ID No 3, Seq ID No 2, Seq ID No 1, and Seq ID No 4, as an OBP.

In a preferred embodiment, the invention relates to the use of a sequence chosen among Seq ID No 3, Seq ID No 1, and Seq ID No 2, as an OBP. Still preferably, the invention relates to the use of a sequence chosen among Seq ID No 3 and Seq ID No 2, as an OBP.

Variants Used as OBPs

The present invention also relates to the use of functional variants of the above described polypeptides as OBPs.

By “functional variant” or “functional variant thereof” it is meant a protein that derives from an OBP according to the invention and that retains at least one domain for the attachment to a hydrophobic compound, preferably an odorant. A functional variant may have a similar or a different binding profile as compared to the OBP of reference. In other word, the functional variant according to the invention may bind the same compounds as the OBP it derives from, but it may also bind only some of these compounds or none of these compounds, and may as well bind other compounds which were not originally bound by the OBP of reference. In particular the functional variant used according to the invention may bind:
(1) Only the same compounds as the OBP it derives from (the OBP of reference);
(2) The same compounds as the OBP it derives from and other compounds which were not originally bound by said OBP of reference;
(3) Only some of the compounds bound by the OBP it derives from;
(4) Some compounds bound by the OBP it derives from and other compounds which were not originally bound by said OBP of reference; or
(5) Only some compounds which were not originally bound by the OBP of reference.

A functional variant used according to the invention includes mutated polypeptides which can exist naturally, in particular in a canine (such as a dog). Modifications can be deletion, addition or substitution of at least one amino acid, a truncation, an elongation and/or a chimeric fusion. By way of example, mention will be made of the possibilities of substitutions capable of being performed without resulting in a change of the biological activities of the polypeptides thereby obtained. These types of substitutions are usually referred to as conservative substitutions. They correspond to the replacement of an amino acid by a different amino acid with similar biochemical properties (e.g., charge, hydrophobicity and/or size). For example, a leucine may be replaced by a valine or an isoleucine; an aspartic acid may be replaced by a glutamic acid; a glutamine by an asparagine, or an arginine by a lysine, etc. Reverse substitutions can also be performed under the same conditions. In particular, it is possible to introduce one or more modifications such as a deletion, addition or substitution of at least one amino acid at the level of the alpha helixes of the protein without impacting the chalice formed by the beta-sheet structure. Similarly, it is possible to introduce equivalent amino acids which can be used to preserve the hydrophobic character to the leaflets beta. It is also commonly known in the art that one conserved tyrosine residue located at the entrance of the binding pocket constitutes the door of the cavity. Structural and molecular dynamics studies combined with mutagenesis experiments have revealed the crucial role of this residue in the uptake of odorants.

In particular, the N-terminal part of the OBP corresponds to a signal peptide, represented in the above sequences Seq ID Nos 1 to 4 in bold characters, whose function is the export the protein outside the cell where it is synthetized. Although the signal peptide has an important function in the OBP, it has no function in the binding capacity of said protein, and, as such, it is not involved in its specificity. It is thus clear that the signal peptide of the isolated protein used according to the invention is optional. Hence, said signal peptide may be absent from the variant or it may be replaced by any other signal peptide, such as a signal peptide which has a similar export function or a signal peptide with a function of addressing said variant to a specific compartment of the cell.

According to the invention, a functional variant of an OBP may be a fragment thereof. In a particular embodiment, a fragment comprises at least the consensus sequence of the OBP it derives from. In a particular embodiment, a fragment comprises at least the amino acids involved in the binding pocket of said OBP. As explained above, a functional variant according to the invention must bind hydrophobic compounds and more preferably odorant compounds. Nevertheless, said compounds may be different from those bound by the OBP of reference (i.e., the OBP said variant derives from).

Advantageously, a functional variant of the isolated protein according to the invention comprises an amino acid sequence having at least 70% of identity with a sequence chosen among Seq ID No 3, Seq ID No 2, Seq ID No 1, and Seq ID No 4. More preferably, the functional variant according to the invention comprises an amino acid sequence having at least 75% of identity, more preferably at least 80% of identity, more preferably at least 85% of identity, more preferably at least 90% of identity, still more preferably at least 91% of identity, still more preferably at least 92% of identity, still more preferably at least 93% of identity, still more preferably at least 94% of identity, still more preferably at least 95% of identity, still more preferably at least 96% of identity, still more preferably at least 97% of identity, still more preferably at least 98% of identity still more preferably at least 99% of identity and still more preferably 100% of identity with a sequence chosen among Seq ID No 1, Seq ID No 2, Seq ID No 3, and Seq ID No 4.

By “identity”, it is meant an exact sequence match between two polypeptides, between two proteins, between two peptides or between two amino acid molecules. The “percentage of identity” or “ID %” between two sequences is a function of the number of identical residues common to both sequences, taking into account the number of intervals that must be introduced for optimal alignment and the length of each interval. Various computer programs and mathematical algorithms are available in the state of the art to determine the percentage of identity between amino acid sequences, such as the Blast program available on the NCBI or ALIGN database (Atlas of Protein Sequence and Structure, Dayhoff (ed.), 1981, Suppl. 3 482-489). For example, “at least 80% sequence identity”, as used here, represents 80%, 81%, 82%, 83%, 84%, 85%, 86%, 87%, 88%, 89%, 90%, 91%, 92%, 93%, 94%, 95%, 96%, 97%, 98%, 99% or 100%.

Consensus Sequences Used as OBPs

For sake of clarity, the consensus sequence of CfamOBP1 is designated by Seq ID No 5, the consensus sequence of CfamOBP2 is designated by Seq ID No 6, the consensus sequence of CfamOBP3 is designated by Seq ID No 7, and the consensus sequence of CfamOBP4 is designated by Seq ID No 8.

By “consensus sequence” it is meant a part of the protein responsible for the function and the binding specificity of the protein and thereby allowing its use as an OBP in the context of the present invention. Notably, the consensus sequence includes the amino acids involved in the formation of the binding pocket of said protein. By “binding pocket” it is meant a cavity on the surface or in the interior of the OBP that possesses suitable properties for binding a ligand, in particular a hydrophobic compound and preferably an odorant compound. In this regard, the 3D structural conformation of the binding pocket is paramount as it is key to the binding specificity of the polypeptide and therefore its use as an OBP. Indeed, its unique configuration allows to adapt to its individual molecular binding partners and facilitates the binding process.

Hence, according to the invention, the OBP preferably comprises a sequence chosen among Seq ID No 7, Seq ID No 6, Seq ID No 5, and Seq ID No 8.

In a preferred embodiment, the invention relates to the use of a sequence chosen among Seq ID No 7, Seq ID No 6, and Seq ID No 5, as an OBP. Still preferably, the invention relates to the use of a sequence chosen among Seq ID No 7 and Seq ID No 6, as an OBP.

According to some embodiments, said OBP further comprises a signal peptide and an alpha helix peptide.

By “signal peptide”, it is meant a peptide used to address a protein to a particular location. This location may be a cell compartment such as the nucleus, endosomes, Golgi or others, it may be a membrane, like a cell membrane or an organelle membrane or it may be outside of the cell, notably for proteins which are secreted. Signal peptides are short peptides, usually comprising 15 to 30 amino acids, and are located in N-terminal or C-terminal position. In particular, all secreted proteins contain a signal peptide which is a hydrophobic sequence of amino acids. In a context of the present invention, the presence of a signal peptide is optional.

By “alpha helix” it is meant a peptide which displays a particular secondary structure which consists in a right hand-helix conformation in which every backbone N—H group hydrogen binds to the backbone C═O group of the amino acid located three or four residues earlier along the protein sequence. Modification of the OBP C-terminal sequence (including the alpha-helix) has no consequence on OBP binding properties ((Ramoni, Bellucci et al. 2007)). Hence, in the context of the invention, the presence of an alpha helix is optional.

In a preferred embodiment, the invention relates to the use of a sequence chosen among Seq ID No 7, Seq ID No 6, Seq ID No 5, and Seq ID No 8, as an OBP, wherein said sequence further comprises an appropriate signal peptide, in particular, a signal peptide which allows to secrete the protein outside the cell where it is synthetized.

In a preferred embodiment, the invention relates to the use of a sequence chosen among Seq ID No 7, Seq ID No 6, Seq ID No 5, and Seq ID No 8 as an OBP, wherein said sequence further comprises an appropriate alpha helix peptide.

In another preferred embodiment, the invention relates to the use as an OBP of a polypeptide according to the invention, comprising:
(i) a sequence chosen among Seq ID No 7, Seq ID No 6, Seq ID No 5, Seq ID No 8, and
(ii) an appropriate signal peptide, in particular a signal peptide which allows to secrete the protein outside the cell where it is synthetized, and
(iii) an appropriate alpha helix peptide.

In another preferred embodiment, the invention relates to the use as an OBP of a polypeptide according to the invention consisting of:
(i) a sequence chosen among Seq ID No 7, Seq ID No 6, Seq ID No 5, Seq ID No 8, and
(ii) an appropriate signal peptide, in particular a signal peptide which allows to secrete the protein outside the cell where it is synthetized, and
(iii) an appropriate alpha helix peptide.

Variants of Consensus Sequences Used as OBPs

The present invention further relates to the use of functional variants of the above identified consensus sequences as OBPs. A “functional variant of a consensus sequence” according to the invention retains at least one domain for the attachment to a hydrophobic compound, preferably an odorant and may have a similar or a different binding profile as compared to the consensus sequence of reference. The definition of “functional variant” given above applies mutatis mutandis.

Hence, in a preferred embodiment, a functional variant of the consensus sequence used as an OBP according to the invention comprises an amino acid sequence having at least 90% of identity with a sequence chosen among Seq ID No 7, Seq ID No 6, Seq ID No 5, and Seq ID No 8. More preferably, the functional variant of the consensus sequence comprises an amino acid sequence having at least 91% of identity, still more preferably at least 92% of identity, still more preferably at least 93% of identity, still more preferably at least 94% of identity, still more preferably at least 95% of identity, still more preferably at least 96% of identity, still more preferably at least 97% of identity, still more preferably at least 98% of identity still more preferably at least 99% of identity and still more preferably 100% of identity with a sequence chosen among Seq ID No 7, Seq ID No 6, Seq ID No 5, and Seq ID No 8.

In some embodiments, the invention relates to the use as an OBP of a functional variant of a sequence having at least 90% of identity with the consensus sequence of any of Seq ID No 3, Seq ID No 2, Seq ID No 1, and Seq ID No 4.

In a preferred embodiment, a functional variant used as an OBP (1) retains at least one domain for the attachment to a hydrophobic compound, preferably an odorant and may have a similar or a different binding profile as compared to the OBP of reference, and/or (2) has an amino acid sequence identity of at least 70% of identity, preferably at least 75% of identity, more preferably at least 80% of identity, more preferably at least 85% of identity, more preferably at least 90% of identity, still more preferably at least 91% of identity, still more preferably at least 92% of identity, still more preferably at least 93% of identity, still more preferably at least 94% of identity, still more preferably at least 95% of identity, still more preferably at least 96% of identity, still more preferably at least 97% of identity, still more preferably at least 98% of identity still more preferably at least 99% of identity and still more preferably 100% of identity with a sequence chosen among Seq ID No 3, Seq ID No 2, Seq ID No 1 and Seq ID No 4, and/or (3) has a sequence identity of at least 90% of identity, preferably at least 91% of identity, still more preferably at least 92% of identity, still more preferably at least 93% of identity, still more preferably at least 94% of identity, still more preferably at least 95% of identity, still more preferably at least 96% of identity, still more preferably at least 97% of identity, still more preferably at least 98% of identity still more preferably at least 99% of identity and still more preferably 100% of identity with a sequence chosen among Seq ID No 5, Seq ID No 6, Seq ID No 7, and Seq ID No 8.

Preferred Variants Used as OBPs

According to some embodiments, a functional variant of Seq ID No 1 is selected among Seq ID No 13 and Seq ID No 14.

According to an embodiment, a functional variant of Seq ID No 2 is Seq ID No 15.

According to an embodiment, a functional variant of Seq ID No 3 is selected among Seq ID No 27, and Seq ID No 35.

According to an embodiment, a functional variant of Seq ID No 4 is selected among Seq ID No 16, and Seq ID No 28.

In a preferred embodiment, the functional variant of the polypeptide used as an OBP according to the invention comprises a sequence chosen among Seq ID No 13, Seq ID No 14, Seq ID No 15, Seq ID No 27, Seq ID No 35, Seq ID No 16, and Seq ID No 28, in particular a sequence chosen among Seq ID No 13, Seq ID No 14, Seq ID No 15, Seq ID No 27, and Seq ID No 35, and still preferably a sequence chosen among Seq ID No 15, Seq ID No 27 and Seq ID No 35. In another embodiment, the functional variant of the polypeptide used as an OBP according to the invention consists in a sequence chosen among Seq ID No 13, Seq ID No 14, Seq ID No 15, Seq ID No 27, Seq ID No 35, Seq ID No 16, and Seq ID No 28, in particular a sequence chosen among Seq ID No 13, Seq ID No 14, Seq ID No 15, Seq ID No 27, and Seq ID No 35, and still preferably a sequence chosen among Seq ID No 15, Seq ID No 27, and Seq ID No 35.

Tag

According to some embodiments, the isolated proteins and polypeptides used as OBPs according to the invention further comprise at least one tag. By “tag” it is meant a peptide sequence which is engrafted in the C-terminal or N-terminal end of a recombinant polypeptide. Tags are notably used in molecular biology for protein purification, location, solubilization or other applications. Tags are numerous and well known in the art. They notably include GST-tag, His-tag, Flag-tag, fluorescent-tag and others.

In another aspect, the present invention relates to a method comprising at least the steps of:
(i) Providing at least one sequence selected in the group consisting of Seq ID No 3, Seq ID No 1, Seq ID No 2, Seq ID No 4, Seq ID No 5, Seq ID No 6, Seq ID No 7, Seq ID No 8, and functional variants thereof; and
(ii) Using said sequence as an OBP.

In this aspect of the invention, the functional variants of step (i) are those described above, and are preferably selected in the group consisting of Seq ID No 13, Seq ID No 14, Seq ID No 15, Seq ID No 27, Seq ID No 35, Seq ID No 16, and Seq ID No 28.

B—Method for Identifying Ligands

In another aspect, the present invention also concerns a method of identifying ligands.

By “ligand” or “ligands” it is meant a chemical compound that binds to an OBP as defined in the present invention and/or forms a complex with it. Preferably, said ligand is a hydrophobic compound, and most preferably odorant compound, wherein the meaning of “hydrophobic compound” and “odorant compounds” is as defined above in the description.

In an embodiment, said method for identifying ligands comprises at least the steps of:
(i) exposing at least one candidate compound to an OBP as defined above or as produced according to the method described below (see Section I “Method for producing an OBP”);
(ii) detecting whether the candidate compound binds to said OBP; and
(iii) if the candidate compound binds to said OBP then the compound is identified as being a ligand to said OBP.

In an alternative step (iii), if the candidate compound does not bind to said OBP then the compound is identified as not being a ligand.

In a particular embodiment, said method comprises at least the following step before said step (i):
Providing at least one sequence selected in the group consisting of Seq ID No 3, Seq ID No 1, Seq ID No 2, Seq ID No 4, Seq ID No 5, Seq ID No 6, Seq ID No 7, Seq ID No 8, and functional variants thereof, wherein said sequence is used as the OBP in step (i).

In this embodiment, the functional variants are those described above, and are preferably selected in the group consisting of Seq ID No 13, Seq ID No 14, Seq ID No 15, Seq ID No 27, Seq ID No 35, Seq ID No 16, and Seq ID No 28.

As used herein, the term “candidate compound” should be understood as a compound whose ability to bind an OBP is yet unknown and is to be tested for first demonstration, or a compound whose ability to bind an OBP is putative and is to be tested for confirmative demonstration.

The candidate compound can be an odorant compound. In a particular embodiment, a composition comprising at least one candidate compound can be used in step (i). For example, said composition can be a food product, a pet food product, a pet food palatability enhancer, a fragrance or a perfume.

In step (ii) a detection method must be employed. Choice of a detection method is well within the skilled person and is for example fluorescence or calorimetry.

Regarding a particular embodiment, the step (ii) of detecting whether the ligand binds to said OBP can be implemented by measuring the fluorescence. Means for measuring a fluorescence are well known in the art, and can notably be implemented by using a spectrofluorometer.

In this favorite embodiment, said method includes a preliminary step (i-1) of contacting said OBP with a fluorescent probe, wherein said fluorescent probe binds the OBP.

Preferably, said fluorescent probes is selected among: 1-N-phenylnaphthylamine (NPN), 1-aminoanthracene (1-AMA), 8-anilinonaphthalene-1-sulfonic acid (1,8-ANS), and SYPRO™ orange. When the fluorescent probe binds the OBP, fluorescent light emission can be detected, notably, in the spectrum comprised between 350 and 700 nm. In particular, emission spectra can be recorded between 350 and 500 nm for NPN; 400 and 600 nm for 1,8-ANS and 510 and 700 nm for SYPRO orange.

Preferably, said fluorescent emission is detected and quantified in order to determine a standard level of fluorescence, corresponding to a state wherein said OBP is bound to said fluorescent probe.

Advantageously, the fluorescent probe binds to the OBP in a reversible manner, so that when the OBP is exposed in step (i) to a ligand, said candidate ligand may chase the fluorescent compound and bind to said OBP.

Preferably the affinity of the fluorescent compound for the OBP ranges from lower than K_{D}=0.1 μM to K_{D}=10 μM ((Zhu, Arena et al. 2017), (Briand, Eloit et al. 2002)). Hence if a candidate compound has an affinity in this range for the OBP ((Mastrogiacomo, D'Ambrosio et al. 2014)), it will release the probe from the OBP cavity (i.e., the OBP binding pocket), and bind within the OBP.

According to this embodiment, when the ligand chases the fluorescent probe and binds to the OBP, a decrease in fluorescence is detected.

By “decrease in the fluorescence” it is meant that the fluorescence decreases as compared to a reference situation. Such a decrease can be of about −1%, preferably −2.5%, preferably −5%, −7.5%, −10%, −15%, −20%, −25%, −30%, −35%, −40%, −45%, −50%, −55%, −60%, −65%, −70%, −75%, −80%, −85%, −90%, −95%, −100% less than the standard level of fluorescence in said reference situation.

Notably, if a shift in the fluorescence is observed in step (ii-2), it can then be concluded in step (iii) that the candidate compound of step (i-3) is a ligand. Alternatively, if no shift in the fluorescence is observed in step (ii-2), it can then be concluded in step (iii) that the candidate compound of step (i-3) is not a ligand.

Hence, in a preferred embodiment, said method for identifying ligands, comprises at least the steps of:
(i-1) exposing at least one fluorescent probe to an OBP;
(i-2) measuring the fluorescent emission to determine a standard level of fluorescence, corresponding to state where an OBP is bound to said fluorescent probe;
(i-3) exposing at least one product to said OBP of step (i-1);
(ii-1) measuring fluorescent emission to determine a second level of fluorescence;
(ii-2) comparing the standard level of fluorescence obtained in step (i-2) to the second level of fluorescence obtained in step (ii-1); and
(iii) if the second level of fluorescence is lower than the standard level of fluorescence then concluding that the candidate compound is a ligand.

Preferably to conclude in step (iii) that the candidate compound is a ligand, the second level of fluorescence determined in step (ii-2) is at least −10%, preferably at least −15%, such as for example −20%, −25%, −30%, −35%, −40%, −45%, −50%, −55%, −60%, −65%, −70%, −75%, −80%, −85%, −90%, −95%, and more preferably −100% lower than the standard level of fluorescence determined in step (vi).

The present invention also relates to the use of an OBP as defined above or as produced according to the method described below (see Section I “Method for producing an OBP”) for identifying ligands, such as odorant compounds, to said OBP. The above-mentioned steps also apply herein.

In another aspect, the present invention relates to the ligand, preferably the odorant compound, identified by the method above.

This method can be used for identifying the OBP binding profile of an OBP.

C—Kits

The present invention also relates to a kit comprising, in one or more containers in a single package:
(i) the OBP as defined in the present invention or obtained by the method described below (see Section I “Method for producing an OBP”), or the polynucleotide described below (see Section E “Isolated polynucleotide”), or the vector described below (see Section F “Vector”), or the recombinant cell described below (see Section G “Host cells”), and
(ii) one or more candidate compounds, such as odorant compounds.

According to the invention “containers” include, but are not limited to, bags, boxes, cartons, bottles, packages of any type or design or material.

The term “single package” means that the elements of said kit are physically associated in or with one or more containers and considered a unit for manufacture, distribution, sale, or use. A single package may be containers of individual elements physically associated such that they are considered a unit for manufacture, distribution, sale, or use.

In particular embodiments according to this aspect of the present invention, the kit further comprises means for communicating information or instructions, to help using the kits' elements.

As used herein, a “means for communicating information or instructions” is a kit element under any form suitable for providing information, instructions, recommendations, and/or warranties, etc. Such a means can comprise a document, digital storage media, optical storage media, audio presentation, visual display containing information. The means of communication can be a displayed web site, brochure, product label, package insert, advertisement, visual display, etc.

In particular, such a kit can be used for identifying odorant compounds, such as using the method according to the present invention.

Advantageously, the kit according to the invention may further comprise an odorant compound known to bind said OBP and to be used as a positive control in said method for identifying odorant compounds. Advantageously, it may also further comprise a compound known not to bind said OBP, to serve as a negative control in said method.

D—Further Methods and Uses of an OBP

The present invention also discloses other methods and uses of a polypeptide as described above, notably a polypeptide as described above chosen among CfamOBP1, CfamOBP2, CfamOBP3, CfamOBP4 and a fragment thereof.

The polypeptide described above or a fragment thereof can also be used in a process for screening compound, preferably hydrophobic compounds, most preferably odorant compounds.

Hence, the invention also relates to a method of detecting the presence of an odorant compound in a composition. Said method comprises at least the steps of:
(i) exposing said composition to an OBP as defined above or as produced according to the method described below, said OBP being known to bind to the odorant compound;
(ii) detecting whether said OBP binds the odorant compound; and
(iii) if the OBP binds the odorant compound, then concluding that the odorant compound is present in said composition.

The composition can be any composition that can be made or used in any kind of industry, including agriculture, and in particular any composition comprising odorant compounds, including a food product, a pet food product, a pet food palatability enhancer or a perfume.

In step (ii) a detection method must be employed. Choice of a detection method is again well within the skilled person and is for example fluorescence or calorimetry. Technical embodiments described in Section B apply herein. Advantageously, methods of identification, such as mass spectrometry, can be also used when the composition comprises multiple odorant compounds susceptible to bind said OBP.

In a particular embodiment, an OBP as defined above may be used in a method for identifying at least one odorant compound in a composition, comprising the steps of:
(i) exposing said composition to an OBP as defined above or as produced according to the method described below;
(ii) detecting whether said OBP binds an odorant compound; and
(iii) identifying said odorant compound.

As above described, the composition can be any composition comprising odorant compounds, including a food product, a pet food product, a pet food palatability enhancer or a perfume.

In step (ii) a detection method must be employed. Choice of a detection method is again well within the skilled person and is for example fluorescence or calorimetry. Technical embodiments described in Section B apply herein.

In step (iii), any method of identification can be employed, such as mass spectrometry.

Said polypeptide described above or fragment thereof may be used as a competitive inhibitor, as an agonist or antagonist of cellular olfactory receptors.

Advantageously, inhibitors of OBP's binding to their specific olfactory receptors may be used in an anti-cancer strategy. Indeed, it is known that some tumors such as breast tumors are hormone-dependent and are sensitive to steroids. Steroids are known to be transported by certain types of lipocalins. Hence, it is within the scope of the invention to provide an inhibitor which prevents the binding of steroids to the polypeptides as described above, in particular to prevent the binding of steroid to a tumoral cell olfactory receptor.

The invention also relates to a pharmaceutical composition comprising a pharmaceutical compound linked to at least one polypeptide as described above and a pharmaceutically acceptable carrier. It is also within the scope of the invention to increase the binding capacity by mutagenesis of the polypeptides according to the invention.

In another embodiment, the polypeptide as described above or a fragment thereof may be used for the preparation of monoclonal or polyclonal antibodies. Monoclonal antibodies can advantageously be prepared from hybridomas according to the technique described by (KÖHler and Milstein 1975). Polyclonal antibodies may be prepared, for example, by immunizing an animal, in particular a mouse, with a polypeptide as described above associated with an adjuvant of the immune response, followed by purification of the specific antibodies contained in the serum of the immunized animals on an affinity column on which the polypeptide that served as antigen has previously been fixed. The polyclonal antibodies according to the invention may also be prepared by purification on an affinity column on which a polypeptide as described above has previously been immobilized, notably a tagged OBP according to the invention.

Hence, the present invention also relates to a monoclonal or polyclonal antibody and fragments thereof, characterized in that they specifically bind a polypeptide as described above. Chimeric antibodies, humanized antibodies and single-chain antibodies are also part of the invention. The antibody fragments according to the invention are preferably Fab or F (ab) 2 fragments.

The invention also relates to a monoclonal antibody specific for a polypeptide as described above and capable of inhibiting the interaction between said polypeptide and the cellular olfactory receptor to which said polypeptide specifically binds. According to another embodiment, the monoclonal antibody according to the invention is capable of inhibiting the interaction between said polypeptide and its hydrophobic ligands, preferably odorant molecules or pheromones with which said polypeptide binds.

The antibodies of the invention may be labeled with an enzymatic, fluorescent or radioactive label. Such labeled antibodies may be used for the detection of these polypeptides in a biological sample. Preferably, the biological sample is a fluid such as serum, blood or human biopsies. They thus constitute a means of analysis of polypeptide expression according to the invention, for example by immunofluorescence, gold labelling, enzyme immunoconjugates.

More generally, the antibodies of the invention can be advantageously used in any situation where the expression of a polypeptide as described above must be observed, and more particularly in immunocytochemistry, immunohistochemistry or in western blotting experiments, in ELISA and RIA techniques. It is thus within the scope of the invention to provide a method for detecting and/or assaying a polypeptide as described above, in a biological sample, characterized in that it comprises the following steps of (i) bringing the biological sample into contact with antibodies according to the invention and then (ii) revealing the antigen-antibody complex formed.

Also included in the scope of the invention is a kit for the detection and/or assay of a polypeptide as described above in a biological sample, characterized in that it comprises the following elements: (i) a monoclonal or polyclonal antibody as described above; (ii) where appropriate, reagents for constituting the medium for the immunological reaction; (iii) reagents for detecting the antigen-antibody complexes produced by the immunological reaction. This kit is particularly useful for the performance of Western blotting experiments and immunoprecipitation.

In yet another embodiment, the polypeptide described above or a fragment thereof can be used in a process for controlling the volatilization of an odorant.

Said process is characterized in that it comprises a first step of binding said odorant with a polypeptide as described above or a fragment thereof.

It is also possible to bind the odorant to a solid support using a polypeptide as described above attached to said support, this binding being able to be both covalent and non-covalent, for example by adsorption or by avidin-biotin-type binders, if necessary. Under these conditions, it is possible to obtain a perfumed support which retains the odors longer, progressively releases the odors and can be used in the cosmetic field as well as in the field of cleaning products in general. Said support may be a plate or a ball type support for example. Of course, the polypeptide as described above will be more particularly usable in the perfumery and cosmetic industry where the polypeptide may be used in the form of a liquid mixture intended to control the volatilization of odors after the composition has been spread on the human skin. This makes it possible to extend perfumes staying power or to be included in the composition of body deodorants in particular. The term “perfume” is used herein in its common meaning and includes in a general way the mixtures known in perfumery, on base of alcohol or in aqueous form, and containing in particular essential oils. The polypeptide as described above may also be used in the composition of retinol-based creams as a retinol transporting and protecting agent for cosmetic applications and in particular to prevent, erase and treat wrinkles and fine lines of the skin, and to combat skin and/or subcutaneous sagging.

Among the applications concerning collective hygiene, the process according to the invention can be used in a device aimed at deodorizing premises such as, for example, pet shops and stables. Such a device can also be used to deodorize the air flow entering an air-conditioning unit; such a device would be very useful in polluted geographical areas.

In another embodiment, the polypeptide as described above or a fragment thereof can be used in a process for solubilizing lipophilic molecules characterized in that it comprises the binding of said lipophilic molecule to a polypeptide as described above.

Indeed, since the OBPs as described above can bind lipophilic molecules thereby solubilizing said lipophilic molecules. Accordingly, the polypeptides as described above or fragments thereof can be used in combination with dietary fatty acids as a food additive.

Furthermore, the polypeptide as described above is likely to be involved in the transport of fatty acids and in the biological mechanisms allowing the detection of the fatty acid load of the food ration, especially at the oral level. The invention therefore also concerns the use of the polypeptides as described above in association with fatty acids to reduce the consumption of fatty acids, particularly in hyperlipidemia or obesity. The polypeptides as described above can therefore be used for the treatment of hyperlipidemia and obesity. Indeed, these proteins participate in the detection of the fatty acid content in the food intake (Gilbertson 1998), an excess of these proteins should lead to lure the physiological system detecting the fat load of a food ration. Thus, a food portion low in fat but supplemented with an OBP as defined above previously loaded with fatty acid will be falsely identified as high in fat.

In yet another embodiment, the polypeptide as described above, or fragment thereof may be used in preventive and curative therapy. In particular, the absence of detection of this type of proteins in a biological sample using an antibody, a primer, a probe according to the invention could be an element of diagnosis of anosmia.

In another embodiment, the polypeptide as described above, or fragment thereof may also be used in pharmaceutical compositions, in particular in order to vectorize certain active drugs. Indeed, OBPs are used by mammals to transport hydrophobic molecules within biological fluids. They even seem to be the natural transporters of xenobiotics including odorants in vivo. For example, an experimental overload of xenobiotics creates tumors in male rats at the proximal bypass tube (TCP) of the nephron (S J Borghoff, B G Short et al. 1990). Indeed, MUP proteins, only produced in male rats, are reabsorbed by TCP cells; after lysosomal degradation, MUP proteins release the xenobiotics they were carrying. These accumulate in these cells and lead them to a tumor pathway by mutagenesis. Lipocalins thus seem to be the ideal structure to trap in their calyx a molecule used as a drug, thus avoiding its general distribution. Thus, (Beste, Schmidt et al. 1999) and the application WO 99 16873 describe a mutagenesis and screening strategy to identify variants of a lipocalin with optimal affinity for a given ligand. It is therefore possible to create a highly specific drug cage, said cage corresponding to the binding pocket of the OBP as described above.

The present invention therefore concerns a polypeptide as described above as a pharmaceutical compound targeting agent. By “targeting agent”, it is meant the polypeptides as described above are able of transporting and releasing the ligand to which they are bound to certain target tissues or cells that have receptors on their surface for the said polypeptides. Thus, the invention relates to drug transport within the binding pocket of a polypeptide as described above and to the targeting of cells through the ability of the polypeptide as described above to bind to a specific olfactory receptor.

It is also within the scope of the invention to develop fusion proteins or chimeric proteins comprising the binding pocket of the polypeptides as described above associated with a protein allowing a specific cell addressing, generally different from the cell where an OBP as defined above is generally addressed. The present invention therefore concerns a polypeptide characterized in that said polypeptide is expressed in the form of a fusion protein with a protein allowing specific cell addressing.

Among the proteins allowing specific cell addressing, interleukins, cytokines, lymphokines, chemokines, growth factors, hormones, mono- or polyclonal antibodies are preferred. Interleukins, cytokines and lymphokines are preferably selected from the group consisting of interleukins Il-1 to Il-20, interferons a-IFN, ss-IFN and y-IFN. Growth factors are preferably colony stimulating factors G-CSF, GM-CSF and erythropoietin. Hormones are preferably selected from steroid hormones.

According to another embodiment, the polypeptide as described above, used as a pharmaceutical compound targeting agent, is associated with a molecule allowing specific cell addressing. Such molecules may be selected from the group of steroids, interleukins, cytokines, lymphokines, interferons, growth factors, hormones and antibodies. Preferably, the molecule is a steroid. The association between the polypeptide as described above and said molecule is achieved either by a non-covalent bond using for example the avidin-biotin system or by a covalent bond using for example chemical bridging agents.

Among the pharmaceutical compounds transported and targeted by the polypeptide as described above are found drugs and in particular anti-cancer agents. Anticancer agents are selected from the group of antiproliferative, antineoplastic or cytotoxic agents and are used to stop the development of cancers and to induce regression and/or elimination of the tumor mass. These anti-cancer agents are preferably radioisotopes, and even more preferably gamma-emitting radioisotopes such as lodel31, Yttrium90.1′Or199, Palladium′00, Copper67, Bismuth2l7 and Antimony2ll. Beta- and alpha-emitting radioisotopes can also be used for therapy. The non-isotopic anti-cancer agents linked to the polypeptide as described above are multiple and varied. They include: (i) antimetabolites such as anti-folate agents, methotrexate, (ii) purine and pyrimidine analogues (mercaptopurine, fluorouracil, 5-azacytidine), (iii) antibiotics, (iv) lectins (ricin, abrin) and (iv) bacterial toxins (diphtheria toxin); the toxins are preferably selected from Pseudomonas exotoxin A, diphtheria toxin, cholera toxin, Bacillus anthrox toxin, Pertussis toxin, Shiga toxin from Shigella, Shiga toxin-like toxin, Escherichia coli toxin, colicin A, d-endotoxin, Haemophilus A hemagglutinin.

The invention also relates to a polypeptide as described above as a pharmaceutical compound transporter. By “transporter”, it is meant that polypeptides as described above capable of transporting a pharmaceutical compound in the organism without the said compound being released at a privileged location in the organism. Such a polypeptide constitutes a means of delivery of said pharmaceutical compound into the organism.

The present invention also relates to a pharmaceutical composition according to the invention characterized in that said polypeptide constitutes a delayed form of delivery of said pharmaceutical compound into the body.

E—Isolated Polynucleotides

Polynucleotides Encoding OBPs

It is also described an isolated polynucleotide encoding at least one OBP, preferably one canine OBP and more particularly those mentioned above.

By “polynucleotide”, “nucleic acid” or “nucleic acid molecule” it is meant a polymer of any length of deoxyribonucleic acid (DNA), or polydeoxyribonucleotides, including but not limited to complementary DNA or cDNA, genomic DNA, plasmids, vectors, viral genomes, isolated DNA, probes, primers and any mixture thereof; or a polymer of any length of ribonucleic acid (RNA), or polyribonucleotides, including in particular messenger RNA or mRNA, antisense RNA; or mixed polyribo-polydeoxyribonucleotides. They include single or double-stranded, linear or circular, natural or synthetic polynucleotides. In addition, a polynucleotide may include non-natural nucleotides and may be interrupted by non-nucleotide components.

The terms “nucleic acid”, “nucleic acid molecule”, “polynucleotide” and “nucleotide sequence” are used herein interchangeably.

It is herein described a polynucleotide encoding at least one of the isolated polypeptides mentioned above (i.e., the OBP and functional variants thereof as described in Section A (Use of isolated proteins and polypeptides as OBPs).

It is also described an isolated polynucleotide comprising at least a sequence chosen among Seq ID No 9, Seq ID No 10, Seq ID No 11, and Seq ID No 12. In an embodiment, the isolated polynucleotide has at least a nucleotide sequence having at least 70% of identity with a sequence chosen among Seq ID No 9, Seq ID No 10, Seq ID No 11, and Seq ID No 12. More preferably, the isolated polynucleotide comprises at least an nucleotide sequence having at least 75% of identity, more preferably at least 80% of identity, more preferably at least 85% of identity, more preferably at least 90% of identity, still more preferably at least 91% of identity, still more preferably at least 92% of identity, still more preferably at least 93% of identity, still more preferably at least 94% of identity, still more preferably at least 95% of identity, still more preferably at least 96% of identity, still more preferably at least 97% of identity, still more preferably at least 98% of identity still more preferably at least 99% of identity and still more preferably 100% of identity with a sequence chosen among Seq ID No 9, Seq ID No 10, Seq ID No 11, Seq ID No 12, and complementary sequences thereof.

By “identity”, it is also meant an exact sequence match between two polynucleotides, between two nucleotide sequences, or between two nucleic acid molecules. The “percentage of identity” or “ID %” between two sequences is a function of the number of identical residues common to both sequences, taking into account the number of intervals that must be introduced for optimal alignment and the length of each interval. Programs to determine the identity between nucleotide sequences are available in a specialized database (e.g., Genbank, the Wisconsin Sequence Analysis Package, the BESTFIT, FASTA and GAP programs). For example, “at least 80% sequence identity”, as used here, represents 80%, 81%, 82%, 83%, 84%, 85%, 86%, 87%, 88%, 89%, 90%, 91%, 92%, 93%, 94%, 95%, 96%, 97%, 98%, 99% or 100%.

In one embodiment, the isolated polynucleotide consists of a sequence chosen among Seq ID No 9, Seq ID No 10, Seq ID No 11, and Seq ID No 12.

In some particular embodiments, the isolated polynucleotide comprises at least a sequence chosen among Seq ID No 17, Seq ID No 18, Seq ID No 19, Seq ID No 20, Seq ID No 21, Seq ID No 22, Seq ID No 23, Seq ID No 24, Seq ID No 25, Seq ID No 26, Seq ID No 29, Seq ID No 30, Seq ID No 31, Seq ID No 32, Seq ID No 33, Seq ID No 34, and Seq ID No 36.

Promoters and Terminators

According to some embodiments, the isolated polynucleotide further comprises at least one transcription promoting sequence. By “transcription promoting sequence”, “transcription promoter” or “promoter” it is meant a nucleotide sequence located near a gene and is essential for the transcription of DNA into RNA. The promoter allows binding of an RNA polymerase before starting RNA synthesis. Promoting sequences are generally located upstream of the transcription start site. There are number of different transcription-promoting sequences, and the skilled artisan knows how to determine which promoter is the most appropriate, in a given context.

According to some embodiments, the isolated polynucleotide further comprises at least one transcription terminator. By “transcription terminator” or “terminator” it is meant a nucleotide sequence that marks the end of the transcription of a gene into messenger RNA by an RNA polymerase. Different transcription terminator sequences exist and the skilled artisan knows how to determine which promoter is the most appropriate, in a given context.

According to a preferred embodiment, the isolated polynucleotide comprises at least the isolated polynucleotide as above defined (in paragraph “Polynucleotides encoding OBPs”), preferably comprising at least a nucleotide sequence encoding a polypeptide or protein chosen among CfamOBP1, CfamOBP2, CfamOBP3, CfamOBP4, and functional variants thereof and further comprises a transcription promoting sequence and a transcription terminator.

According to another preferred embodiment, the isolated polynucleotide comprises at least a nucleotide sequence encoding a polypeptide having at least 70% of identity with a sequence chosen among Seq ID No 3, Seq ID No 2, Seq ID No 1, and Seq ID No 4, and further comprises a transcription promoting sequence and a transcription terminator.

According to yet another preferred embodiment, the isolated polynucleotide comprises at least a nucleotide sequence having at least 70% of identity with a sequence chosen among Seq ID No 9, Seq ID No 10, Seq ID No 11, and Seq ID No 12, and further comprises a transcription promoting sequence and a transcription terminator.

Tag

According to some embodiments, the isolated polynucleotide further comprises a nucleic sequence encoding at least one tag. Sequences coding for said tags are numerous and well known in the art, and may be located at either end (5′ end and/or 3′ end) of the isolated polynucleotide.

Type of Polynucleotides

In an embodiment, the polynucleotide is isolated from a mammalian cell, notably from a canine cell, and preferably from a dog cell.

In an embodiment, the polynucleotide is isolated from a vector or a host cell comprising said polynucleotide, said vector or host cell being as defined and described below in sections G and F “Host Cells” or “Vectors”.

The isolated polynucleotide can be synthesized in-vitro by nucleic acid synthesis techniques which are well known to the person skilled in the art or can be determined without difficulty from his or her general knowledge.

According to a method of production, the isolated nucleic acid molecule is recombinant.

TABLE 1
summary of Seq ID correspondence

| | | Sequence |
|---|---|---|
| Seq ID No | Sequence description | type |
| Seq ID No 1 | CfamOBP1 | Protein |
| Seq ID No 2 | CfamOBP2 | Protein |
| Seq ID No 3 | CfamOBP3 | Protein |
| Seq ID No 4 | CfamOBP4 | Protein |
| Seq ID No 5 | CfamOBP1 Consensus sequence | Protein |
| Seq ID No 6 | CfamOBP2 Consensus sequence | Protein |
| Seq ID No 7 | CfamOBP3 Consensus sequence | Protein |
| Seq ID No 8 | CfamOBP4 Consensus sequence | Protein |
| Seq ID No 9 | CfamOBP1 DNA seq > NM_001003190.1 | DNA |
| Seq ID No 10 | CfamOBP2 DNA seq > NM_001003189.2 | DNA |
| Seq ID No 11 | CfamOBP3 DNA seq > NM_001284461.1 | DNA |
| Seq ID No 12 | CfamOBP4 DNA seq > XM_843825.5 | DNA |
| Seq ID No 13 | CfamOBP1 variant Pro21Thr | Protein |
| Seq ID No 14 | CfamOBP1 variant Ser52Leu | Protein |
| Seq ID No 15 | CfamOBP2 variant Glu83Lys | Protein |
| Seq ID No 16 | CfamOBP4 variant Ser140Gly | Protein |
| Seq ID No 17 | CfamOBP1 variant A61C | DNA |
| Seq ID No 18 | CfamOBP1 variant C155T | DNA |
| Seq ID No 19 | CfamOBP1 variant G399A | DNA |
| Seq ID No 20 | CfamOBP2 variant G51A | DNA |
| Seq ID No 21 | CfamOBP2 variant T225C | DNA |
| Seq ID No 22 | CfamOBP2 variant G247A | DNA |
| Seq ID No 23 | CfamOBP2 variant T409C | DNA |
| Seq ID No 24 | CfamOBP2 variant T462C | DNA |
| Seq ID No 25 | CfamOBP3 variant G231A | DNA |
| Seq ID No 26 | CfamOBP4 variant A418G | DNA |
| Seq ID No 27 | CfamOBP3 variant Ile71Val | Protein |
| Seq ID No 28 | CfamOBP4 variant Asn164Asp | Protein |
| Seq ID No 29 | CfamOBP1 variant G363A | DNA |
| Seq ID No 30 | CfamOBP3 variant G99A | DNA |
| Seq ID No 31 | CfamOBP3 variant A211G | DNA |
| Seq ID No 32 | CfamOBP4 variant C12T | DNA |
| Seq ID No 33 | CfamOBP4 variant C177T | DNA |
| Seq ID No 34 | CfamOBP4 variant A490G | DNA |
| Seq ID No 35 | CfamOBP3 mutant 102Y | Protein |
| Seq ID No 36 | CfamOBP3 mutant 102Y | DNA |

F—Vectors

It is also herein described an expression vector comprising the polynucleotide as above defined.

By “vector” it is meant a vehicle, preferably a nucleic acid molecule or a viral particle, which contains the necessary elements to enable the administration, propagation and/or expression of one or more nucleic acid molecule(s) in a host cell or organism.

Functionally, this term includes vectors for maintenance (cloning vectors), vectors for expression in various host cells or organisms (expression vectors), extrachromosomal vectors (e.g., multicopy plasmids) or integration vectors (e.g., designed to integrate into the genome of a host cell and produce additional copies of the nucleic acid molecule contained therein when the host cell replicates). The term also includes shuttle vectors (e.g., operating in both prokaryotic and/or eukaryotic hosts) and transfer vectors (e.g., for the transfer of nucleic acid molecule(s) into the genome of a host cell).

Structurally, vectors can be natural, synthetic, or a combination of natural and artificial genetic elements.

The term “vector” as used herein is to be understood broadly to include plasmid and viral vectors.

A “plasmid” as used herein refers to a replicable DNA construct. Typically, plasmid vectors contain selection marker genes which allow host cells carrying the plasmid to be identified and/or selected positively or negatively in the presence of the compound corresponding to the selection marker. A variety of positive and negative selection marker genes are known in the art. For example, an antibiotic resistance gene can be used as a positive selection marker gene to select a host cell in the presence of the corresponding antibiotic.

The term “viral vector” as used herein refers to a nucleic acid vector that comprises at least one element of a virus genome and can be packaged in a viral particle. Viral vectors may be replication competent or selective (e.g., designed to replicate better or selectively in specific host cells), or may be genetically inactivated so as to be defective or deficient for replication.

Advantageously, the vector is a plasmid.

Vectors suitable in the present context include, without limitation, bacteriophage, plasmid or cosmid vectors for expression in prokaryotic host cells such as bacteria (e.g. E. coli, Bacillus or bacteria of the genus Pseudomonas); vectors for expression in yeast (e.g. Saccharomyces cerevisiae, Schyzosaccharomyces pombe, Pichia pastoris); baculovirus vectors for expression in insect cell systems (e.g. Sf 9 cells); viral and plasmid vectors for expression in plant cell systems (e.g. plasmid Ti, Cauliflower Mosaic Virus, CaMV, Tobacco Mosaic Virus TMV); and viral and plasmid vectors for expression in cells or higher eukaryotic organisms.

By “bacterium” or “bacteria” it is meant a microscopic, prokaryotic organism(s) present in a given medium. Preferred bacteria used herein are of the species Escherichia coli.

By “yeast” it is meant a microscopic, eukaryotic organism(s) some species of which are capable of causing the fermentation of organic matter. Preferred yeast used herein are of the genus Pichia.

Vectors suitable in the present context may be of the integrative or replicative type. Vectors of the integrative type do not have a sequence known as the “origin of replication” and must therefore be integrated directly into the genome of the host cell in order to be expressed. This integration can be done using dedicated genetic tools that are well known to the profession. As an example, this integration can be carried out by homologous recombination or via the CRE-LOX recombination system. Replicative vectors have an autonomous replication origin. These vectors therefore replicate independently of the host cell genome. Thus, unlike integrative vectors, replicating vectors do not need to be integrated into the host cell genome.

These vectors are generally available commercially (e.g., from suppliers such as Invitrogen, Promega, etc.), available from depositary institutions such as the American Type Culture Collection (ATCC, Rockville, Md.), or have been the subject of numerous publications describing their sequence, structures and production methods, so that the person in the field can apply them without difficulty.

Representative examples of suitable bacterial plasmid vectors include pQE-30, pQE-31, pET vectors.

Representative examples of suitable yeast plasmid vectors include pPIC9 and pPIC3.5K.

G—Host Cells

It is also herein described a recombinant cell expressing, preferably overexpressing, an OBP as defined above.

By “recombinant cell” or “host cell”, it is meant a cell containing at least one exogenous nucleic acid molecule. According to the invention, the recombinant cell can be a prokaryotic cell, such as a bacterium, or a eukaryotic cell, such as a yeast, an insect cell or a mammalian cell. Advantageously, the recombinant cell expresses, preferably overexpresses, at least one odorant binding protein or a functional variant thereof.

Thus, a recombinant cell is not a naturally occurring cell but is a molecular biology tool obtained by genetic manipulation techniques.

By “exogenous” genetic material or sequence means that the said genetic material or sequence originates from another organism, which may belong or not to the same cell line.

By “overexpression”, “overexpressing” it is meant that the cell expression of a gene or of a protein, yet notably an OBP gene or protein, is increased compared to a reference situation. Such an overexpression can be of about +1%, preferably +2.5%, preferably +5%, +7.5%, +10%, +15%, +20%, +25%, +30%, +35%, +40%, +45%, +50%, +55%, +60%, +65%, +70%, +75%, +80%, +85%, +90%, +95%, +100% higher than the standard level of expression in said reference situation. The one of ordinary skill in the art knows appropriate methods and techniques to assess an increased expression level compared to a standard expression level. By “overexpression”, “overexpressing” it is also meant that a cell that normally does not express a gene or a protein, in particular an OBP gene or protein, now expresses said gene or a protein.

According to various embodiments, said host cell can be a prokaryotic cell, or an eukaryotic cell such as a yeast cell, or another eukaryotic cell such as an insect, plant or mammalian cell (e.g., human or non-human, preferably non-human cells). Advantageously, the host cell is a bacterium, especially of the species Escherichia coli, or a yeast, notably a yeast of the genus Pichia.

The recombinant cell preferably comprises at least one polynucleotide or at least one vector as defined above. Preferably, said recombinant cell is stably transformed with the vector as above defined.

By “transformed” or “transformation” it is meant the introduction of exogenous genetic material into prokaryotic cells, in particular those used herein. For the sake of simplification, in the present description, the terms “transfected” or “transfection” and “transformed” or “transformation” are used interchangeably.

A recombinant cell or host cell is “stably transformed” when the exogenous genetic material is stably introduced into the cell so that said exogenous genetic material may be expressed continuously, and may also be expressed in later generations of said cell. Several methods for obtaining a stable transformation exist and are known in the art. Notably the exogenous genetic material may be integrated directly into the host cell genome through an integrative vector. Alternatively, the exogenous material may be integrated into a replicative vector that will replicate independently of the host cell genome. Preferably a selection marker is used and transfected together with the exogenous genetic material to maintain a selection pressure on the transformed cell and avoid the loss the exogenous genetic material.

Hence, it is further described a host cell stably transformed with at least one copy of a polynucleotide encoding at least a polypeptide as mentioned above (i.e., the OBP and functional variants thereof as described in Section A “Use of isolated proteins and polypeptides as OBPs”, in particular encoding at least a polypeptide chosen among CfamOBP1, CfamOBP2, CfamOBP3, CfamOBP4, and functional variants thereof.

Preferably, the host cell is stably transformed with at least one copy of a polynucleotide as defined above (in paragraph “Polynucleotides encoding OBPs”), in particular a polynucleotide having at least a sequence chosen among Seq ID No 9, Seq ID No 10, Seq ID No 11, and Seq ID No 12.

In some embodiments, the host cell is stably transformed with at least one copy of a polynucleotide having at least a sequence chosen among Seq ID No 17, Seq ID No 18, Seq ID No 19, Seq ID No 20, Seq ID No 21, Seq ID No 22, Seq ID No 23, Seq ID No 24, Seq ID No 25, Seq ID No 26, Seq ID No 29, Seq ID No 30, Seq ID No 31, Seq ID No 32, Seq ID No 33, Seq ID No 34, and Seq ID No 36.

H—Method for Obtaining a Recombinant Cell Expressing an OBP

It is also herein described a method for obtaining a recombinant cell expressing, preferably overexpressing an OBP, as defined above.

In a preferred embodiment, said method for obtaining a recombinant cell comprises at least the steps of:
(i) providing a polynucleotide as defined above;
(ii) cloning said polynucleotide provided in step (i) in a vector capable of expressing said molecule in a cell;
(iii) contacting said cell and said vector obtained in step (ii) under appropriate conditions so that the former is transformed, preferably stably transformed, by said vector and so that said cell expresses said polynucleotide, said cell thus being recombinant.

Said polynucleotide of step (i) encodes at least a polypeptide as mentioned above (i.e., the OBP and functional variants thereof as described in Section A “Use of isolated proteins and polypeptides as OBPs”, in particular encoding at least a polypeptide chosen among CfamOBP1, CfamOBP2, CfamOBP3, CfamOBP4, and functional variants thereof.

In an embodiment, said polynucleotide of step (i) encodes a polypeptide having at least an amino acid sequence chosen among Seq ID No 13, Seq ID No 14, Seq ID No 15, Seq ID No 16, Seq ID No 27, Seq ID No 35 and Seq ID No 28.

In yet another preferred embodiment, said polynucleotide is as defined above (in paragraph “Polynucleotides encoding OBPs”), in particular a said polynucleotide has a sequence chosen among Seq ID No 9, Seq ID No 10, Seq ID No 11, and Seq ID No 12.

In an embodiment, said polynucleotide of step (i) has at least a sequence chosen among Seq ID No 17, Seq ID No 18, Seq ID No 19, Seq ID No 20, Seq ID No 21, Seq ID No 22, Seq ID No 23, Seq ID No 24, Seq ID No 25, Seq ID No 26, Seq ID No 29, Seq ID No 30, Seq ID No 31, Seq ID No 32, Seq ID No 33, Seq ID No 34, and Seq ID No 36. Preferably, said polynucleotide of step (i) further comprises a promoting sequence and a transcription terminator, as defined above.

According to an embodiment, said polynucleotide is integrated in step (iii) directly into the host cell genome. In an alternative embodiment, the polynucleotide is integrated into a replicative vector that replicates independently of the host cell genome.

According to various embodiments and as described above, said host cell can be a prokaryotic cell, or a eukaryotic cell such as a lower eukaryotic cell, notably a yeast cell, or another eukaryotic cell such as an insect, plant or mammalian cell (e.g., human or non-human, preferably non-human cells). Preferably, the host cell is a bacterium, notably an Escherichia coli strain.

I—Method for Producing an OBP

The present description also concerns a method for producing an OBP, notably an OBP as defined above.

In a preferred embodiment, said method for producing an OBP comprises at least the steps of:
(i) providing a recombinant cell as defined above or obtaining a recombinant cell by carrying out the above-described method;
(ii) culturing said recombinant cell in an appropriate culture medium under suitable conditions to produce an OBP;
(iii) recovering said OBP either in said recombinant cell or in the culture medium as obtained after step (ii) and optionally purifying said OBP.

Advantageously, the recombinant cell is as described above. In particular, it is preferably a bacteria cell, especially of the species Escherichia coli. According to other embodiments, said recombinant cell is a yeast, notably a yeast of the genus Pichia.

Based on his or her general knowledge, the skilled person will be able to identify the culturing conditions adapted to produce an OBP according to step (ii). In particular, he or she will know how to adjust the temperature, pH, amount of O₂ and CO₂, and other parameters, depending on the cell type used in the implementation of the production method described above.

Depending on the presence or absence of a signal peptide, said OBP will be produced intracellularly or extracellularly (in the culture medium). In some embodiments, it is produced in the periplasm when bacteria are used. Preferably, the OBP produced by the method described above is engrafted with a tag which facilitates the optional protein purification of step (iii). Methods of protein purification are well known in the art; they notably include affinity chromatography, steric exclusion chromatography, ion exchange chromatography.

The following examples are intended to illustrate the present invention without any limitation.

### EXAMPLES

### Example 1: Material and Methods

### 1.1. Collection of the Nasal Mucus Samples

Olfactory mucus was collected from six dogs under general anaesthesia by a veterinary. The breeds studied were American Staff (1 male, 1 female), Belgian Shepherd (2 males, 1 female) and a Setter (1 male).

Collection of samples was achieved during an anaesthesia for other purpose than the withdrawal, in accordance with the protocol and did not cause discomfort to the animals.

In both nostrils, olfactory mucus was collected with a cotton soaked with physiological serum.

Sample collection was performed under the approval of Diana Petfood's ethic committee.

### 1.2. Olfactory Mucus Analysis by LC/MS

Proteins were separated using Sodium dodecyl sulfate polyacrylamide gel electrophoresis (12% acrylamide) (SDS-PAGE) and digested using trypsin. The separation of the protein digests was done using an UltiMate 300 rapid separation liquid chromatography (RSLC) Nano System (Thermo Fisher Scientific, USA). Peptide fractionation was automatically done onto a commercial C18 reversed-phase column (75 μm×250 mm, 2-μm particle, PepMap100 RSLC column, Thermo Fisher Scientific (USA) at 35° C.). A solvent containing 98% H₂O, 2% acetonitrile and 0.1% formic acid was used for trapping, which was done in 4 min at a flowrate of 5 μL/min. The elution was performed using two solvents, A (0.1% formic acid in water) and B (0.1% formic acid in acetonitrile), at a flow rate of 300 nL/min. The separation gradient was 3 min at 3% followed by 110 min from 3% to 20% B, then 10 min from 20% to 80% B that is maintained for 15 min. Prior to each experiment the column is equilibrated during 6 min with 3% of buffer B. Analysis of the eluted peptides was done using Q-Extractive instruments (Thermo Fisher Scientific, USA). Electrospray voltage was set at 1.9 kV and capillary temperature set at 275° C. Full mass spectrometry scan was collected in the Orbitrap mass analyzer over the m/z 300-1200 range with a resolution of 35,000 (m/z 200). Target value was 3.00E+06. The fifteen most intense peaks within these spectra, with charge states between 2 and 5 were fragmented. Mass spectrum was acquired in the Orbitrap mass analyser with a resolution of 17,500 at m/z 200. Target value was 1.00E+05. Maximum allowed ion accumulation times were 250 ms for full MS scans and 100 ms for tandem mass spectra. Ion selection threshold was 5.0E+04 counts. Dynamic exclusion was set at 30 s.

### 1.3. Strains and Material

The coding sequence for the four CfamOBPs were extracted from Uniprot database (https://www.uniprot.org/). The identification numbers are O18873 (CfamOBP1), O18874 (CfamOBP2), H2B3G5 (CfamOBP3) and E2R1I1 (CfamOBP4). CfamOBPs coding sequences were optimized for expression in E. coli, synthesized by Genewiz (Germany), and cloned into the plasmid pQE31 from Qiagen (Netherlands). The plasmid coding for the mutant of Seq ID No 35 was synthetized by site directed mutagenesis on CfamOBP3 by Genewiz (Germany). The coding sequences were inserted between BamHI and HindIII restriction sites included in pQE31 plasmid. E. coli M15 were then transformed with the plasmid through heat shock.

### 1.4. Protein Production and Purification

LB medium was used for the production of CfamOBP1 and its variant (Seq ID No 14) and CfamOBP3 and its variant (Seq ID No 35) while Terrific Broth (TB) medium was used for CfamOBP2 and its variant (Seq ID No 15) and CfamOBP4 production. Except CfamOBP1, which was produced at 29° C., all the proteins were produced at 37° C. All of the cultures were done in presence of 0.1 mg/mL ampicillin and 0.025 mg/mL kanamycin. Protein expression was induced by 0.2 mM isopropyl-β-D-thiogalactoside (IPTG) when DO₆₀₀ₙₘ was comprised between 0.5 and 0.7. After induction, cells were grown during 6 additional hours. Then, cell growth was stopped by centrifugation at 4,000 g during 20 min. The cell pellets were resuspended in buffer containing NaH₂PO₄ 50 mM, NaCl 300 mM, Imidazole 20 mM at pH 7.5. Resuspended cells were disrupted by sonication at 4° C. (pulse 5 s, rest 3 s, 60 W) followed by 45 min centrifugation at 20,000 g. The centrifugation supernatant was loaded 3 times successively on a His-Trap HP column (GE Healthcare, USA). After a combination of washing using different buffers as previously described protocol ((Brute, Glaz et al. 2020)), CfamOBPs were eluted by a linear gradient using an increasing concentration of a buffer containing NaH₂PO₄ 50 mM, NaCl 300 mM, Imidazole 250 mM at pH 7.5.

Proteins were then dialyzed in 4 successive baths containing 100 mM phosphate buffer at pH 7.5 added to 5% acetonitrile for the two firsts.

The purified proteins concentrations were then quantified by UV spectroscopy using molar extinction coefficients at 280 nm of 13,075 M⁻¹·cm⁻¹, 24,075 M⁻¹·cm⁻¹, 16,180 M⁻¹·cm⁻¹ and 17,545 M⁻¹·cm⁻¹ for CfamOBP1, CfamOBP2, CfamOBP3 and CfamOBP4, respectively. The molar extinction coefficients at 280 nm are not modified for the variants. The molar extinction coefficients were calculated from the amino acid sequence on ProtParam (https://web.expasy.org/protparam/).

### 1.5. Recombinant OBP Characterization

SDS-PAGE with Coomassie blue staining were performed using a Mini-Protean II system (Bio-Rad, France). The Precision Plus Protein Dual Xtra Standards was used as a reference on the gels for the molecular masses (Bio-rad, France).

### 1.6 Fluorescence Binding Assays

Binding experiments were performed at 20° C. on a spectrofluorometer (Cary-Eclipse (Agilent, USA)) equipped with magnetic stirrers and a Peltier control temperature unit. The fluorescent probes: 1-N-phenylnaphthylamine (NPN) (Sigma, USA), 8-anilinonaphthalene-1-sulfonic acid (1,8-ANS) (Sigma, USA), and SYPRO™ orange (ThermoFischer, France) were excited at 337 nm, 372 nm and 490 nm respectively. Emission spectra were recorded between 350 and 500 nm for NPN; 400 and 600 nm for 1,8-ANS and 510 and 700 nm for SYPRO orange. Fluorescent probe binding experiments were performed in a 2 mL cuvette of 1 cm path length containing 2 μM OBPs in 50 mM potassium phosphate buffer at pH 7.5. The fluorescent probes were prepared in 100% MeOH at 0.1, 1, 10 and 100 mM. The concentration of the different fluorescent probes was increased into the 1 mL solution containing CfamOBPs until the adding allow to reach a fluorescence maximum of emission intensity. The binding curves were then traced using SigmaPlot 12.5 software.

Ligand competitive binding experiments were performed in cuvettes containing 2 μM OBPs, 10 μM fluorescent probe for CfamOBP1 and its variant (Seq ID No 14) and CfamOBP3, 4 μM fluorescent probe for CfamOBP2 and its variant (Seq ID No 15) and 1.25× fluorescent probe for CfamOBP4, in 50 mM potassium phosphate buffer at pH 7.5.

Odorants were prepared in 100% MeOH and aliquots were added to final concentrations of 0.5-20 μM. The experiments were performed in triplicate. The obtained data were then traced and analysed using SigmaPlot 12.5 software.

### 1.7 Isothermal Titration Calorimetry

Titration experiments were carried at 25° C. using isothermal titration microcalorimeter VP-ITC system (Malvern Instruments, Malvern, UK). 100 mM odorant solution in MeOH were diluted to 250 μM into 100 mM potassium phosphate buffer, pH 7.5 and loaded into the syringe. Protein solutions were prepared at 25 μM in the phosphate buffer and degassed directly prior to experiment and injected into the measure cell. Injections of 10 μL ligand solution into the protein containing cell were performed at time intervals of 210 s with a total of 25 injections. Analysis of the experimental data was performed using the Origin 5.0 program (Malvern Instruments, Malvern, UK) and fitted by a model of one binding site. Binding parameters such as stoichiometry (n) and the association constant (KA) were determined by this fitting.

### Example 2: Results

### 2.1. Identification of Dog Odorant Binding Proteins

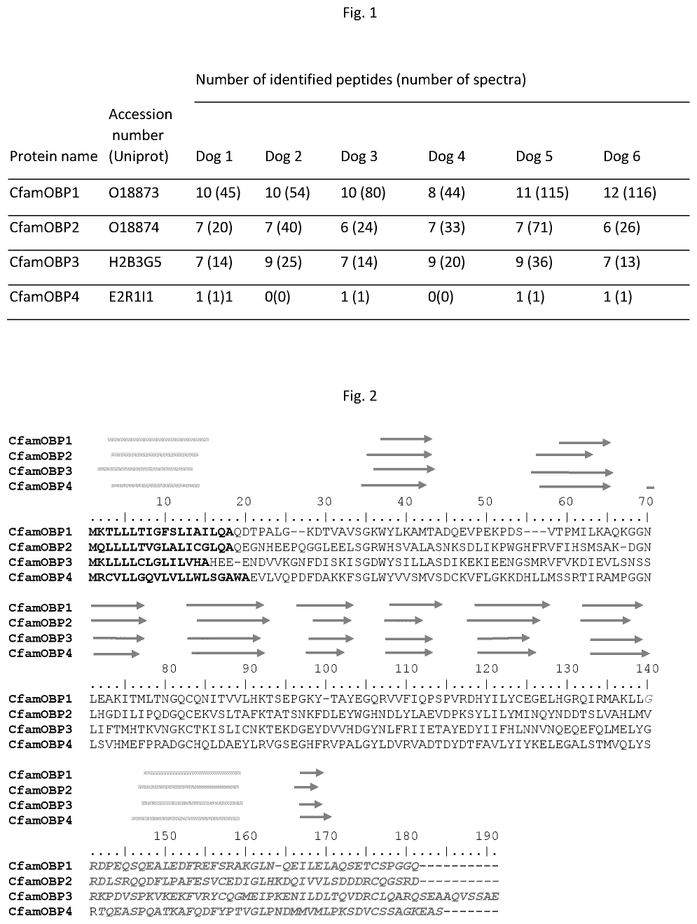

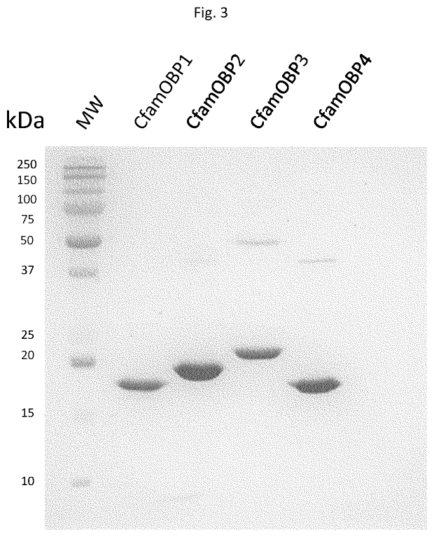

Nasal mucus samples from six dogs were analysed on GC/MS allowing to identified a total of 2,245 different proteins, counting 1,195, 1,184, 1,537, 1,235, 1,234, and 1,285 proteins for each dog, respectively. Among the identified proteins, four were referenced on Uniprot database as lipocalins. As expected, these proteins are typically small (160-180 amino acid residues) and contain the GxW motif typical of lipocalins ((Flower 1996), (Pelosi 1998)). Based on our biochemical characterization, four proteins among the identified lipocalins have been renamed CfamOBP1 (O18873), CfamOBP2 (O18874) and CfamOBP3 (H2B3G5), CfamOBP4 (E2R1I1) (FIG. 1). Their amino acid sequences have been extracted and analysed using Signal P 5.0 (http://www.cbs.dtu.dk/services/SignalP/) in order to predict the sequence corresponding to the peptidic signal (FIG. 2). The secondary structures have been predicted from the previously solved structures of CfamOBP2 and CfamOBP3. Concerning CfamOBP1 and CfamOBP4, the secondary structures have been predicted using Jpred4 software (http://www.compbio.dundee.ac.uk/jpred/). The four proteins present the eight characteristic β-sheets previously described into the lipocalins.

### 2.2. Production and Purification of Dog Odorant Binding Proteins

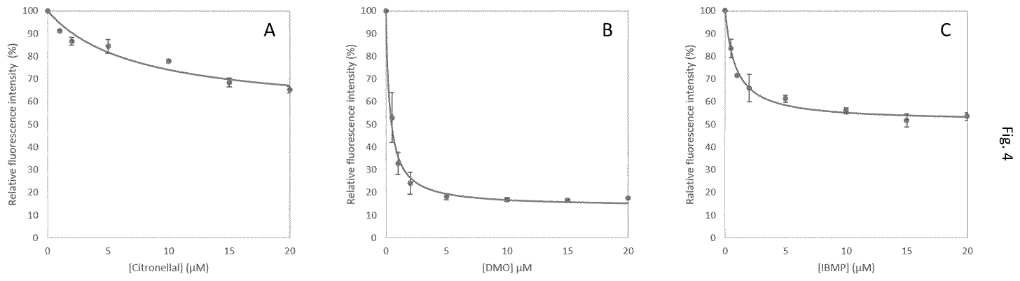

CfamOBPs were heterologously expressed in the E. coli M15 strain after transformation with the corresponding plasmids, cultivation at 29 or 37° C. and induction. The recombinant proteins were then purified using immobilized metal affinity chromatography and a linear gradient of imidazole. For CfamOBP1, CfamOBP3 and its variant of Seq ID No 35 an average of 10 mg of purified protein per liter of bacterial culture was obtained. For CfamOBP2 and its variant (Seq ID No 15) a higher quantity of 30 mg of purified protein per liter of bacterial culture was obtained. Variant of Seq ID No 14 and CfamOBP4 are produced at lower levels with 4 mg of purified protein per liter of bacterial culture. SDS-PAGE analysis (FIG. 3) of the four OBPs shows a single band corresponding at an estimated mass of 19, 20, 21, and 19 kDa for CfamOBP1, CfamOBP2, CfamOBP3 and CfamOBP4, respectively.

### 2.3. Fluorescence Binding Studies

In presence of the protein, the emission maximum of the probes shifts to smaller wavelength and higher intensity level, which will decrease with the addition of ligands of the OBPs.

For each protein, the fluorescent probe showing the highest emission fluorescent intensity was determined. Thereby three different probes have been selected: 1,8-ANS for CfamOBP1 and variant of Seq ID No 14, NPN for CfamOBP2, CfamOBP3 and variant of Seq ID No 15 and SYPRO orange for CfamOBP4. Maximum fluorescence intensity was recorded at 470 nm, 400 nm, 420 nm and 600 nm for CfamOBP1/variant of Seq ID No 14, CfamOBP2/variant of Seq ID No 15, CfamOBP3 and CfamOBP4, respectively.

The tested odorant 3,7-dimethyoctanol (DMO) was previously used on a previous study conducted on dog nasal mucus (D'Auria, Staiano et al. 2006) and 2-Isobutyl-3-methoxypyrazine (IBMP) is a previously described odorant on OBPs ((Briand, Eloit et al. 2002), (Lobel, Strotmann et al. 2001), (Pelosi, Baldaccini et al. 1982)).

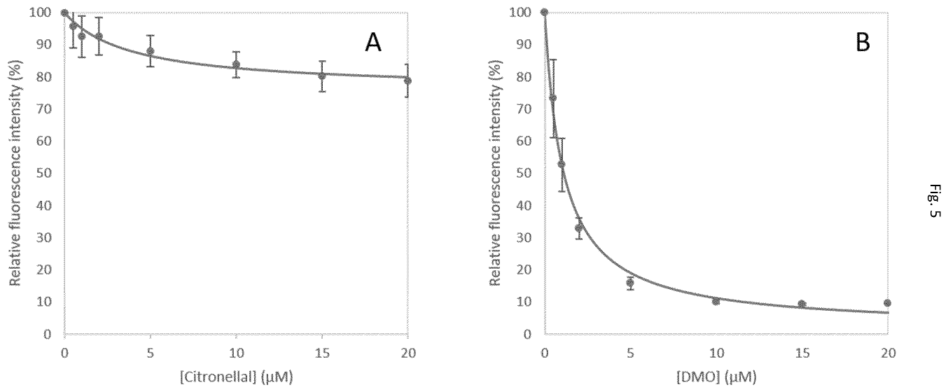

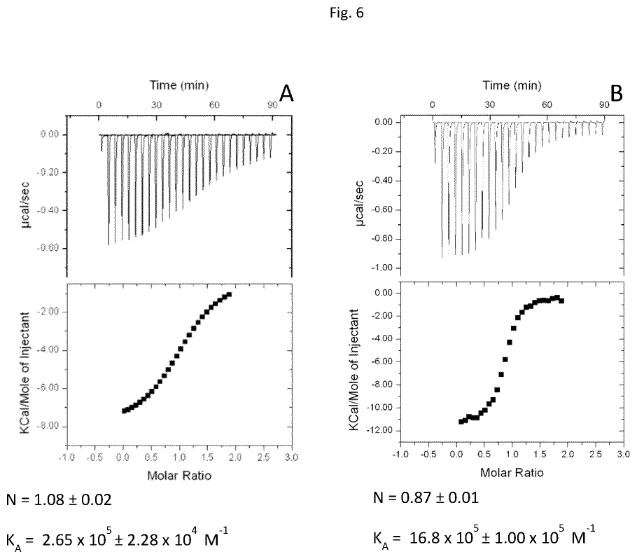

Interestingly citronellal, DMO and IBMP were able to displace the probes for CfamOBP1/variant of Seq ID No 14, CfamOBP2/Seq ID No 15, and CfamOBP3, respectively (FIGS. 4 and 5).

These results support the function as OBP of these different lipocalins identified in the olfactory mucus.

### 2.4. Isothermal Titration Calorimetry

CfamOBP3 having the particularity to possess a histidine residue at the entrance of the binding cavity instead of a conserved tyrosine residue, the impact of this residue substitution on OBP binding properties was analysed with the mutant of CfamOBP3 (Seq ID No 35). The binding properties were studied using ITC with the previously identified ligand IBMP. Mutant of Seq ID No 35 exhibited a dramatically increase of affinity for IBMP with a eight fold increase of its Ka value. This result supports the crucial role of the histidine residue in the binding properties of CfamOBP3.

### BIBLIOGRAPHY

Beste, G., et al. (1999). “Small antibody-like proteins with prescribed ligand specificities derived from the lipocalin fold.” Proc Natl Acad Sci USA 96(5): 1898-1903.
Briand, L., et al. (2002). “Evidence of an odorant-binding protein in the human olfactory mucus: location, structural characterization, and odorant-binding properties.” Biochemistry 41(23): 7241-7252.
Brute, M., et al. (2020). “Bacterial expression and purification of vertebrate odorant-binding proteins.” Methods Enzymol 642: 125-150.
D'Auria, S., et al. (2006). “The odorant-binding protein from Canis familiaris: purification, characterization and new perspectives in biohazard assessment.” Protein Pept Lett 13(4): 349-352.
Flower, D. R. (1996). “The lipocalin protein family: structure and function.” Biochem J 318 (Pt 1): 1-14.
Gilbertson, T. A. (1998). “Gustatory mechanisms for the detection of fat.” Curr Opin Neurobiol 8(4): 447-452.
KÖHler, G. and C. Milstein (1975). “Continuous cultures of fused cells secreting antibody of predefined specificity.” Nature 256(5517): 495-497.
Lobel, D., et al. (2001). “Identification of a third rat odorant-binding protein (OBP3).” Chem Senses 26(6): 673-680.
Mastrogiacomo, R., et al. (2014). “An odorant-binding protein is abundantly expressed in the nose and in the seminal fluid of the rabbit.” PLOS ONE 9(11): e111932.
Pelosi, P. (1998). “Odorant-binding proteins: structural aspects.” Ann N Y Acad Sci 855: 281-293.
Pelosi, P., et al. (1982). “Identification of a specific olfactory receptor for 2-isobutyl-3-methoxypyrazine.” Biochem J 201(1): 245-248.
Ramoni, R., et al. (2007). “The protein scaffold of the lipocalin odorant-binding protein is suitable for the design of new biosensors for the detection of explosive components.” Journal of Physics: Condensed Matter 19(39): 395012.
S J Borghoff, et al. (1990). “Biochemical Mechanisms and Pathobiology of α2u-Globulin Nephropathy.” Annual Review of Pharmacology and Toxicology 30(1): 349-367.
Tegoni, M., et al. (2004). “Structural aspects of sexual attraction and chemical communication in insects.” Trends Biochem Sci 29(5): 257-264.
Zhu, J., et al. (2017). “Reverse chemical ecology: Olfactory proteins from the giant panda and their interactions with putative pheromones and bamboo volatiles.” Proc Natl Acad Sci USA 114(46): E9802-e9810.

1. A method comprising the step of:
use a sequence chosen among Seq ID No 3, Seq ID No 1, Seq ID No 2, Seq ID No 4, Seq ID No 5, Seq ID No 6, Seq ID No 7, Seq ID No 8, and functional variants thereof, as an odorant binding protein (OBP).

2. A method of identifying ligands, comprising at least the steps of:
(i) exposing at least one candidate compound to an OBP as defined in claim 1;
(ii) detecting whether the candidate compound binds to said OBP; and
(iii) if the candidate compound binds to said OBP then the compound is identified as being a ligand to said OBP.

3. The method according to claim 2, comprising at least the following step before said step (i):
Providing at least one sequence selected in the group consisting of Seq ID No 3, Seq ID No 1, Seq ID No 2, Seq ID No 4, Seq ID No 5, Seq ID No 6, Seq ID No 7, Seq ID No 8, and functional variants thereof, wherein said sequence is used as the OBP in step (i).

4. A method of detecting the presence of an odorant compound in a composition, comprising at least the steps of:
(i) exposing said composition to an OBP as defined in claim 1;
(ii) detecting whether said OBP binds the odorant compound; and
(iii) if the OBP binds the odorant compound, then concluding that the odorant compound is present in said composition.

5. A kit comprising, in one or more containers in a single package:
(i) the OBP as defined in claim 1, and
(ii) one or more candidate compounds.


SEQUENCE LISTING (36 sequences)

SEQ ID NO: 1 - protein, 174 aa, Artificial
  CfamOBP1
MKTLLLTIGFSLIAILQAQDTPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 2 - protein, 180 aa, Artificial
  CfamOBP2
MQLLLLTVGLALICGLQAQEGNHEEPQGGLEELSGRWHSVALASNKSDLIKPWGHFRVFIHSMSAKDGNLHGDILIPQDGQCEKVSLTAFKTATSNKFDLEYWGHNDLYLAEVDPKSYLILYMINQYNDDTSLVAHLMVRDLSRQQDFLPAFESVCEDIGLHKDQIVVLSDDDRCQGSRD

SEQ ID NO: 3 - protein, 190 aa, Artificial
  CfamOBP3
MKLLLLCLGLILVHAHEEENDVVKGNFDISKISGDWYSILLASDIKEKIEENGSMRVFVKDIEVLSNSSLIFTMHTKVNGKCTKISLICNKTEKDGEYDVVHDGYNLFRIIETAYEDYIIFHLNNVNQEQEFQLMELYGRKPDVSPKVKEKFVRYCQGMEIPKENILDLTQVDRCLQARQSEAAQVSSAE

SEQ ID NO: 4 - protein, 184 aa, Artificial
  CfamOBP4
MRCVLLGQVLVLLWLSGAWAEVLVQPDFDAKKFSGLWYVVSMVSDCKVFLGKKDHLLMSSRTIRAMPGGNLSVHMEFPRADGCHQLDAEYLRVGSEGHFRVPALGYLDVRVADTDYDTFAVLYIYKELEGALSTMVQLYSRTQEASPQATKAFQDFYPTVGLPNDMMVMLPKSDVCSSAGKEAS

SEQ ID NO: 5 - protein, 115 aa, Artificial
  CfamOBP1 Consensus sequence
QDTPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLL

SEQ ID NO: 6 - protein, 121 aa, Artificial
  CfamOBP2 Consensus sequence
QEGNHEEPQGGLEELSGRWHSVALASNKSDLIKPWGHFRVFIHSMSAKDGNLHGDILIPQDGQCEKVSLTAFKTATSNKFDLEYWGHNDLYLAEVDPKSYLILYMINQYNDDTSLVAHLMV

SEQ ID NO: 7 - protein, 124 aa, Artificial
  CfamOBP3 Consensus sequence
HEEENDVVKGNFDISKISGDWYSILLASDIKEKIEENGSMRVFVKDIEVLSNSSLIFTMHTKVNGKCTKISLICNKTEKDGEYDVVHDGYNLFRIIETAYEDYIIFHLNNVNQEQEFQLMELYG

SEQ ID NO: 8 - protein, 121 aa, Artificial
  CfamOBP4 Consensus sequence
EVLVQPDFDAKKFSGLWYVVSMVSDCKVFLGKKDHLLMSSRTIRAMPGGNLSVHMEFPRADGCHQLDAEYLRVGSEGHFRVPALGYLDVRVADTDYDTFAVLYIYKELEGALSTMVQLYSR

SEQ ID NO: 9 - DNA, 525 bp, Artificial
  CfamOBP1 DNA seq >NM_001003190.1 Canis lupus familiaris odorant binding protein 2B (OBP2B), mRNA
atgaagaccctgctcctcaccatcggcttcagcctcattgcgatcctgcaggcccaggataccccagccttgggaaaggacactgtggctgtgtcagggaaatggtatctgaaggccatgacagcagaccaggaggtgcctgagaagcctgactcagtgactcccatgatcctcaaagcccagaaggggggcaacctggaagccaagatcaccatgctgacaaatggtcagtgccagaacatcacggtggtcctgcacaaaacctctgagcctggcaaatacacggcatacgagggccagcgtgtcgtgttcatccagccgtccccggtgagggaccactacattctctactgcgagggcgagctccatgggaggcagatccgaatggccaagcttctgggaagggatcctgagcagagccaagaggccttggaggattttcgggaattctcaagagccaaaggattgaaccaggagattttggaactcgcgcagagcgaaacctgctctccaggaggacagtag

SEQ ID NO: 10 - DNA, 543 bp, Artificial
  CfamOBP2 DNA seq >NM_001003189.2 Canis lupus familiaris minor allergen Can f 2 (CANF2),mRNA
atgcagctcctactgctgaccgtgggcctggcactgatctgtggcctccaggctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatccttataccgcaggacggccagtgcgagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacttgatggtccgggacctcagcaggcagcaggacttcctgccggcattcgaatctgtatgtgaagacatcggtctgcacaaggaccagattgtggttctgagcgatgacgatcgctgccagggttccagagactag

SEQ ID NO: 11 - DNA, 573 bp, Artificial
  CfamOBP3 DNA seq >NM_001284461.1 Canis lupus familiaris allergen Fel d 4-like (LOC481674), mRNA
atgaagctgctgttgctgtgtctggggctgattctagtccatgcccacgaggaagaaaacgatgttgtgaaaggaaacttcgatatttcaaagatttcgggagattggtattccattctcttggcctcagatatcaaggaaaagatagaagaaaatggcagcatgagggtttttgtgaaagacattgaagtcctgagcaactcttctctgatctttacaatgcatacaaaggtgaatgggaagtgtactaaaatttctctgatttgtaacaaaacagaaaaggatggtgaatatgatgttgtgcatgatggatacaatttatttagaataattgaaacagcctatgaggactatattatatttcatcttaataatgtcaaccaggaacaggaattccaactgatggagctctatggccgaaaaccagatgtgagtccaaaagtcaaggaaaagtttgtgagatattgccaaggaatggaaattcctaaggaaaacatacttgacctgacccaagttgatcgctgtctccaggcccgacagagcgaagcagcccaggtctccagtgctgagtga

SEQ ID NO: 12 - DNA, 555 bp, Artificial
  CfamOBP4  DNA seq >XM_843825.5 PREDICTED Canis lupus familiaris lipocalin 15 (LCN15), transcript variant X3, mRNA
atgaggtgtgtcctgctgggccaggtcctggtgctgctctggctgtccggggcttgggctgaggtcctggtacagccagattttgatgccaaaaagttctcaggtctttggtacgtggtctccatggtctccgactgcaaggtcttcctgggcaagaaggaccacttgctcatgtccagcaggactatcagggccatgccagggggcaacctcagtgtccacatggagttccctcgggctgacggctgtcaccagctggatgctgagtacctgagggtgggctctgaggggcacttcagagtcccagccctgggctacctggacgtgcgcgtggcagacacagactatgacaccttcgccgtgctctacatctataaggagctggagggggcgctcagcaccatggtccagctctacagccggacccaggaagcaagtccccaagccacaaaggccttccaggacttctaccccaccgtggggctccccaatgacatgatggtcatgctgcccaagtcagatgtgtgctcctctgcaggcaaggaggcttcctga

SEQ ID NO: 13 - protein, 174 aa, Artificial
  CfamOBP1 variant Pro21Thr
MKTLLLTIGFSLIAILQAQDPPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 14 - protein, 174 aa, Artificial
  CfamOBP1 variant Ser52Leu
MKTLLLTIGFSLIAILQAQDTPALGKDTVAVSGKWYLKAMTADQEVPEKPDLVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 15 - protein, 180 aa, Artificial
  CfamOBP2 variant Glu83Lys
MQLLLLTVGLALICGLQAQEGNHEEPQGGLEELSGRWHSVALASNKSDLIKPWGHFRVFIHSMSAKDGNLHGDILIPQDGQCKKVSLTAFKTATSNKFDLEYWGHNDLYLAEVDPKSYLILYMINQYNDDTSLVAHLMVRDLSRQQDFLPAFESVCEDIGLHKDQIVVLSDDDRCQGSRD

SEQ ID NO: 16 - protein, 184 aa, Artificial
  CfamOBP4 variant variant Ser140Gly
MRCVLLGQVLVLLWLSGAWAEVLVQPDFDAKKFSGLWYVVSMVSDCKVFLGKKDHLLMSSRTIRAMPGGNLSVHMEFPRADGCHQLDAEYLRVGSEGHFRVPALGYLDVRVADTDYDTFAVLYIYKELEGALSTMVQLYGRTQEASPQATKAFQDFYPTVGLPNDMMVMLPKSDVCSSAGKEAS

SEQ ID NO: 17 - DNA, 525 bp, Artificial
  CfamOBP1 variant A61C
atgaagaccctgctcctcaccatcggcttcagcctcattgcgatcctgcaggcccaggatcccccagccttgggaaaggacactgtggctgtgtcagggaaatggtatctgaaggccatgacagcagaccaggaggtgcctgagaagcctgactcagtgactcccatgatcctcaaagcccagaaggggggcaacctggaagccaagatcaccatgctgacaaatggtcagtgccagaacatcacggtggtcctgcacaaaacctctgagcctggcaaatacacggcatacgagggccagcgtgtcgtgttcatccagccgtccccggtgagggaccactacattctctactgcgagggcgagctccatgggaggcagatccgaatggccaagcttctgggaagggatcctgagcagagccaagaggccttggaggattttcgggaattctcaagagccaaaggattgaaccaggagattttggaactcgcgcagagcgaaacctgctctccaggaggacagtag

SEQ ID NO: 18 - DNA, 525 bp, Artificial
  CfamOBP1 variant C155T
atgaagaccctgctcctcaccatcggcttcagcctcattgcgatcctgcaggcccaggataccccagccttgggaaaggacactgtggctgtgtcagggaaatggtatctgaaggccatgacagcagaccaggaggtgcctgagaagcctgacttagtgactcccatgatcctcaaagcccagaaggggggcaacctggaagccaagatcaccatgctgacaaatggtcagtgccagaacatcacggtggtcctgcacaaaacctctgagcctggcaaatacacggcatacgagggccagcgtgtcgtgttcatccagccgtccccggtgagggaccactacattctctactgcgagggcgagctccatgggaggcagatccgaatggccaagcttctgggaagggatcctgagcagagccaagaggccttggaggattttcgggaattctcaagagccaaaggattgaaccaggagattttggaactcgcgcagagcgaaacctgctctccaggaggacagtag

SEQ ID NO: 19 - DNA, 525 bp, Artificial
  CfamOBP1 variant G399A
atgaagaccctgctcctcaccatcggcttcagcctcattgcgatcctgcaggcccaggataccccagccttgggaaaggacactgtggctgtgtcagggaaatggtatctgaaggccatgacagcagaccaggaggtgcctgagaagcctgactcagtgactcccatgatcctcaaagcccagaaggggggcaacctggaagccaagatcaccatgctgacaaatggtcagtgccagaacatcacggtggtcctgcacaaaacctctgagcctggcaaatacacggcatacgagggccagcgtgtcgtgttcatccagccgtccccggtgagggaccactacattctctactgcgagggcgagctccatgggaggcagatccgaatggccaagcttctaggaagggatcctgagcagagccaagaggccttggaggattttcgggaattctcaagagccaaaggattgaaccaggagattttggaactcgcgcagagcgaaacctgctctccaggaggacagtag

SEQ ID NO: 20 - DNA, 543 bp, Artificial
  CfamOBP2 variant G51A
atgcagctcctactgctgaccgtgggcctggcactgatctgtggcctccaagctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatccttataccgcaggacggccagtgcgagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacttgatggtccgggacctcagcaggcagcaggacttcctgccggcattcgaatctgtatgtgaagacatcggtctgcacaaggaccagattgtggttctgagcgatgacgatcgctgccagggttccagagactag

SEQ ID NO: 21 - DNA, 543 bp, Artificial
  CfamOBP2 variant T225C
atgcagctcctactgctgaccgtgggcctggcactgatctgtggcctccaggctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatcctcataccgcaggacggccagtgcgagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacttgatggtccgggacctcagcaggcagcaggacttcctgccggcattcgaatctgtatgtgaagacatcggtctgcacaaggaccagattgtggttctgagcgatgacgatcgctgccagggttccagagactag

SEQ ID NO: 22 - DNA, 543 bp, Artificial
  CfamOBP2 variant G247A
atgcagctcctactgctgaccgtgggcctggcactgatctgtggcctccaggctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatccttataccgcaggacggccagtgcaagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacttgatggtccgggacctcagcaggcagcaggacttcctgccggcattcgaatctgtatgtgaagacatcggtctgcacaaggaccagattgtggttctgagcgatgacgatcgctgccagggttccagagactag

SEQ ID NO: 23 - DNA, 543 bp, Artificial
  Cfam OBP2 variant T409C
atgcagctcctactgctgaccgtgggcctggcactgatctgtggcctccaggctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatccttataccgcaggacggccagtgcgagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacctgatggtccgggacctcagcaggcagcaggacttcctgccggcattcgaatctgtatgtgaagacatcggtctgcacaaggaccagattgtggttctgagcgatgacgatcgctgccagggttccagagactag

SEQ ID NO: 24 - DNA, 543 bp, Artificial
  CfamOBP2 variant T462C
atgcagctcctactgctgaccgtgggcctggcactgatctgtggcctccaggctcaggagggaaaccatgaggagccccagggaggcctagaggagctgtctgggaggtggcactccgttgccctggcctccaacaagtccgatctgatcaaaccctgggggcacttcagggttttcatccacagcatgagcgcaaaggacggcaacctgcacggggatatccttataccgcaggacggccagtgcgagaaagtctccctcactgcgttcaagactgccaccagcaacaaatttgacctggagtactggggacacaatgacctgtacctggcagaggtagaccccaagagctacctgattctctacatgatcaaccagtacaacgatgacaccagcctggtggctcacttgatggtccgggacctcagcaggcagcaggacttcctgccggcattcgaatccgtatgtgaagacatcggtctgcacaaggaccagattgtggttctgagcgatgacgatcgctgccagggttccagagactag

SEQ ID NO: 25 - DNA, 573 bp, Artificial
  CfamOBP3 variant G231A
atgaagctgctgttgctgtgtctggggctgattctagtccatgcccacgaggaagaaaacgatgttgtgaaaggaaacttcgatatttcaaagatttcgggagattggtattccattctcttggcctcagatatcaaggaaaagatagaagaaaatggcagcatgagggtttttgtgaaagacattgaagtcctgagcaactcttctctgatctttacaatgcatacaaaagtgaatgggaagtgtactaaaatttctctgatttgtaacaaaacagaaaaggatggtgaatatgatgttgtgcatgatggatacaatttatttagaataattgaaacagcctatgaggactatattatatttcatcttaataatgtcaaccaggaacaggaattccaactgatggagctctatggccgaaaaccagatgtgagtccaaaagtcaaggaaaagtttgtgagatattgccaaggaatggaaattcctaaggaaaacatacttgacctgacccaagttgatcgctgtctccaggcccgacagagcgaagcagcccaggtctccagtgctgagtga

SEQ ID NO: 26 - DNA, 555 bp, Artificial
  CfamOBP4 variant A418G
atgaggtgtgtcctgctgggccaggtcctggtgctgctctggctgtccggggcttgggctgaggtcctggtacagccagattttgatgccaaaaagttctcaggtctttggtacgtggtctccatggtctccgactgcaaggtcttcctgggcaagaaggaccacttgctcatgtccagcaggactatcagggccatgccagggggcaacctcagtgtccacatggagttccctcgggctgacggctgtcaccagctggatgctgagtacctgagggtgggctctgaggggcacttcagagtcccagccctgggctacctggacgtgcgcgtggcagacacagactatgacaccttcgccgtgctctacatctataaggagctggagggggcgctcagcaccatggtccagctctacggccggacccaggaagcaagtccccaagccacaaaggccttccaggacttctaccccaccgtggggctccccaatgacatgatggtcatgctgcccaagtcagatgtgtgctcctctgcaggcaaggaggcttcctga

SEQ ID NO: 27 - protein, 190 aa, Artificial
  CfamOBP3 variant Ile71Val
MKLLLLCLGLILVHAHEEENDVVKGNFDISKISGDWYSILLASDIKEKIEENGSMRVFVKDIEVLSNSSLVFTMHTKVNGKCTKISLICNKTEKDGEYDVVHDGYNLFRIIETAYEDYIIFHLNNVNQEQEFQLMELYGRKPDVSPKVKEKFVRYCQGMEIPKENILDLTQVDRCLQARQSEAAQVSSAE

SEQ ID NO: 28 - protein, 184 aa, Artificial
  CfamOBP4 variant Asn164Asp
MRCVLLGQVLVLLWLSGAWAEVLVQPDFDAKKFSGLWYVVSMVSDCKVFLGKKDHLLMSSRTIRAMPGGNLSVHMEFPRADGCHQLDAEYLRVGSEGHFRVPALGYLDVRVADTDYDTFAVLYIYKELEGALSTMVQLYSRTQEASPQATKAFQDFYPTVGLPDDMMVMLPKSDVCSSAGKEAS

SEQ ID NO: 29 - DNA, 525 bp, Artificial
  CfamOBP1 variant G363A
atgaagaccctgctcctcaccatcggcttcagcctcattgcgatcctgcaggcccaggataccccagccttgggaaaggacactgtggctgtgtcagggaaatggtatctgaaggccatgacagcagaccaggaggtgcctgagaagcctgactcagtgactcccatgatcctcaaagcccagaaggggggcaacctggaagccaagatcaccatgctgacaaatggtcagtgccagaacatcacggtggtcctgcacaaaacctctgagcctggcaaatacacggcatacgagggccagcgtgtcgtgttcatccagccgtccccggtgagggaccactacattctctactgcgagggcgaactccatgggaggcagatccgaatggccaagcttctgggaagggatcctgagcagagccaagaggccttggaggattttcgggaattctcaagagccaaaggattgaaccaggagattttggaactcgcgcagagcgaaacctgctctccaggaggacagtag

SEQ ID NO: 30 - DNA, 573 bp, Artificial
  CfamOBP3 variant G99A
atgaagctgctgttgctgtgtctggggctgattctagtccatgcccacgaggaagaaaacgatgttgtgaaaggaaacttcgatatttcaaagatttcaggagattggtattccattctcttggcctcagatatcaaggaaaagatagaagaaaatggcagcatgagggtttttgtgaaagacattgaagtcctgagcaactcttctctgatctttacaatgcatacaaaggtgaatgggaagtgtactaaaatttctctgatttgtaacaaaacagaaaaggatggtgaatatgatgttgtgcatgatggatacaatttatttagaataattgaaacagcctatgaggactatattatatttcatcttaataatgtcaaccaggaacaggaattccaactgatggagctctatggccgaaaaccagatgtgagtccaaaagtcaaggaaaagtttgtgagatattgccaaggaatggaaattcctaaggaaaacatacttgacctgacccaagttgatcgctgtctccaggcccgacagagcgaagcagcccaggtctccagtgctgagtga

SEQ ID NO: 31 - DNA, 573 bp, Artificial
  CfamOBP3 variant A211G
atgaagctgctgttgctgtgtctggggctgattctagtccatgcccacgaggaagaaaacgatgttgtgaaaggaaacttcgatatttcaaagatttcgggagattggtattccattctcttggcctcagatatcaaggaaaagatagaagaaaatggcagcatgagggtttttgtgaaagacattgaagtcctgagcaactcttctctggtctttacaatgcatacaaaggtgaatgggaagtgtactaaaatttctctgatttgtaacaaaacagaaaaggatggtgaatatgatgttgtgcatgatggatacaatttatttagaataattgaaacagcctatgaggactatattatatttcatcttaataatgtcaaccaggaacaggaattccaactgatggagctctatggccgaaaaccagatgtgagtccaaaagtcaaggaaaagtttgtgagatattgccaaggaatggaaattcctaaggaaaacatacttgacctgacccaagttgatcgctgtctccaggcccgacagagcgaagcagcccaggtctccagtgctgagtga

SEQ ID NO: 32 - DNA, 555 bp, Artificial
  CfamOBP4 variant C12T
atgaggtgtgttctgctgggccaggtcctggtgctgctctggctgtccggggcttgggctgaggtcctggtacagccagattttgatgccaaaaagttctcaggtctttggtacgtggtctccatggtctccgactgcaaggtcttcctgggcaagaaggaccacttgctcatgtccagcaggactatcagggccatgccagggggcaacctcagtgtccacatggagttccctcgggctgacggctgtcaccagctggatgctgagtacctgagggtgggctctgaggggcacttcagagtcccagccctgggctacctggacgtgcgcgtggcagacacagactatgacaccttcgccgtgctctacatctataaggagctggagggggcgctcagcaccatggtccagctctacagccggacccaggaagcaagtccccaagccacaaaggccttccaggacttctaccccaccgtggggctccccaatgacatgatggtcatgctgcccaagtcagatgtgtgctcctctgcaggcaaggaggcttcctga

SEQ ID NO: 33 - DNA, 555 bp, Artificial
  CfamOBP4 variant C177T
atgaggtgtgtcctgctgggccaggtcctggtgctgctctggctgtccggggcttgggctgaggtcctggtacagccagattttgatgccaaaaagttctcaggtctttggtacgtggtctccatggtctccgactgcaaggtcttcctgggcaagaaggaccacttgctcatgtctagcaggactatcagggccatgccagggggcaacctcagtgtccacatggagttccctcgggctgacggctgtcaccagctggatgctgagtacctgagggtgggctctgaggggcacttcagagtcccagccctgggctacctggacgtgcgcgtggcagacacagactatgacaccttcgccgtgctctacatctataaggagctggagggggcgctcagcaccatggtccagctctacagccggacccaggaagcaagtccccaagccacaaaggccttccaggacttctaccccaccgtggggctccccaatgacatgatggtcatgctgcccaagtcagatgtgtgctcctctgcaggcaaggaggcttcctga

SEQ ID NO: 34 - DNA, 555 bp, Artificial
  CfamOBP4 variant A490G
atgaggtgtgtcctgctgggccaggtcctggtgctgctctggctgtccggggcttgggctgaggtcctggtacagccagattttgatgccaaaaagttctcaggtctttggtacgtggtctccatggtctccgactgcaaggtcttcctgggcaagaaggaccacttgctcatgtccagcaggactatcagggccatgccagggggcaacctcagtgtccacatggagttccctcgggctgacggctgtcaccagctggatgctgagtacctgagggtgggctctgaggggcacttcagagtcccagccctgggctacctggacgtgcgcgtggcagacacagactatgacaccttcgccgtgctctacatctataaggagctggagggggcgctcagcaccatggtccagctctacagccggacccaggaagcaagtccccaagccacaaaggccttccaggacttctaccccaccgtggggctccccgatgacatgatggtcatgctgcccaagtcagatgtgtgctcctctgcaggcaaggaggcttcctga

SEQ ID NO: 35 - protein, 190 aa, Artificial
  CfamOBP3 mutant 102Y
MKLLLLCLGLILVHAHEEENDVVKGNFDISKISGDWYSILLASDIKEKIEENGSMRVFVKDIEVLSNSSLIFTMHTKVNGKCTKISLICNKTEKDGEYDVVYDGYNLFRIIETAYEDYIIFHLNNVNQEQEFQLMELYGRKPDVSPKVKEKFVRYCQGMEIPKENILDLTQVDRCLQARQSEAAQVSSAE

SEQ ID NO: 36 - DNA, 564 bp, Artificial
  CfamOBP3 mutant 102Y
atgagaggatctcaccatcaccatcaccatacggatccgcatgaggaggaaaacgacgttgtgaagggcaacttcgacatcagcaagatcagcggcgattggtacagcattctgctggcgagcgacatcaaggagaagatcgaggagaatggcagcatgcgcgtgttcgtgaaggacatcgaagtgctcagcaacagcagtctgatcttcacgatgcacaccaaggtgaacggcaaatgcaccaaaatcagcctcatttgcaacaagaccgagaaggacggcgagtacgatgtggtgtatgatggttacaacctcttccgcatcatcgagacggcctacgaagactacatcatcttccatctcaacaacgtgaatcaagaacaagaattccagctgatggagctgtacggccgcaaaccggatgtgagcccaaaggtgaaggagaagttcgtgcgctactgtcaaggcatggagatcccgaaagagaacattctggatctgacgcaagttgaccgctgtctgcaagcccgccaaagcgaagccgcgcaagtgagcagcgccgaa